(19) United States
(12) Patent Application Publication
(10) Pub. No.: US 2023/0277642 A1
(43) Pub. Date: Sep. 7, 2023

(54) LIVE SALMONELLA TYPHI VECTORS ENGINEERED TO EXPRESS CANCER PROTEIN ANTIGENS AND METHODS OF USE THEREOF

(71) Applicants: University of Maryland, Baltimore, Baltimore, MD (US); IRAZÚ Bio LLC, Baltimore, MD (US)

(72) Inventors: James E. Galen, Eldersburg, MD (US); Thanh Pham, Elkridge, MD (US); Marco Chacon, Bel Air, MD (US)

(21) Appl. No.: 17/902,986

(22) Filed: Sep. 5, 2022

Related U.S. Application Data
(63) Continuation-in-part of application No. PCT/US2021/021202, filed on Mar. 5, 2021.
(60) Provisional application No. 62/985,699, filed on Mar. 5, 2020.

(51) Int. Cl. A61K 39/00; C12N 15/74; A61P 35/00

(52) U.S. Cl. CPC A61K 39/001182; A61K 39/00117; A61P 35/00; C12N 15/74; A61K 2039/523; A61K 2039/6006; A61K 2039/627; A61K 2039/70

(57) ABSTRACT

The present invention provides compositions and methods for inducing an immune response in a subject in need thereof, comprising administering to the subject an immunologically-effective amount of a live Salmonella Typhi vector, wherein the Salmonella Typhi vector has been engineered to express one or more cancer antigens. In some aspects the vector has been engineered to express an outer membrane folding protein BamA or a fragment or variant thereof; and a lipid A deacylase PagL or a fragment or variant thereof, wherein the Salmonella Typhi vector is capable of delivering the antigen to a mucosal tissue or subcutaneously to dendritic cells via an outer membrane vesicle when administered to a subject.

### INCORPORATION-BY-REFERENCE OF MATERIAL SUBMITTED ELECTRONICALLY

Incorporated by reference in its entirety herein is a computer-readable sequence listing and identified as follows: One 66,734 Byte XML file named “Sequence_listing.xml,” created on May 30, 2023.

### FIELD OF THE INVENTION

The field of the invention relates generally to the field of medicine, cancer, and molecular biology, in particular vaccine immunotherapy technology.

### BACKGROUND OF THE INVENTION

Cancer is the second leading cause of death in the United States (behind heart disease), with colorectal cancer (CRC) ranking among the top three carcinogenic causes of morbidity and mortality for 2019 in both men and women of the United States (Siegel et al., CA Cancer J Clin 2019; 69(1): 7-34; Miller et al., CA Cancer J Clin 2019; 69(5): 363-85). Conventional treatment therapies for colon, rectal, and anal cancers typically include surgical resection, radiation, and/or chemotherapy involving an ever-expanding array of constantly improving classes of compounds (Libutti et al., Philadelphia: Wolters Kluwer Health; 2016; Libutti et al., Philadelphia: Wolters Kluwer Health; 2016; Czito et al., Philadelphia: Wolters Kluwer Health; 2016). Until recently, less aggressive treatment strategies attempting to exploit the host immune system to clear tumor tissues seemed out of reach. However, with the recent success of immune checkpoint inhibitors that activate tumor-specific T cells, thereby circumventing immunosuppressive mechanisms of the tumor microenvironment to enhance tumor clearance, interest in immunotherapeutic approaches to the treatment of cancer now engenders increasing optimism (Lynch et al., Annals of translational medicine 2016; 4(16): 305). In addition to dampening immunosuppression of T cells using immune checkpoint inhibitors, efforts are also underway to enhance innate immunity through activation of dendritic cells, providing cross-presentation of tumor-associated antigens (TAAs) to cytotoxic T cells, as well as recruitment of T cell help to augment both T cell-mediated and antibody-dependent cell-mediated cytotoxicity (ADCC) responses (Wculek et al., Nat Rev Immunol 2019.).

Salmonella has been one of the organisms most studied for use as a mucosal live carrier vaccine delivering foreign antigens to the immune system. Over the years, several attenuated vaccine strains of Salmonella derived from serovar Typhi have been developed (Tacket et al., Infect Immun 1997; 65(2): 452-6; Wang et al., Infect Immun 2000; 68(8): 4647-52; Wang et al., Infect Immun 2001; 69(8): 4734-41; Tacket et al., Infect Immun 2000; 68: 1196-201). Some attenuated vaccine strains have elicited a broad array of immune responses in clinical trials including intestinal secretory IgA antibodies, serum IgG antibodies, and T cell mediated immunity (Tacket et al., Infect Immun 1997; 65(2): 452-6.; Tacket et al., Infect Immun 2000; 68: 1196-201). The ability of live orally administered S. Typhi to activate circulating human monocyte and dendritic cells, and to prime CD8⁺ T cells through dendritic cell cross presentation in humans has recently been confirmed (Toapanta et al., PLoS Negl TropDis 2015; 9(6): e0003837; Salerno-Goncalves et al., PLoS One 2009; 4(6): e5879).

Use of Salmonella for the therapeutic intervention of cancer in humans has been tested in clinical trials using both S. Typhimurium and S. Typhi serovars, with disappointing results. However, efficacy in humans using Salmonella strains depends on the serovar being used; S. Typhi is adapted to the human host and can penetrate deep into human tissues after oral administration, while S. Typhimurium is rapidly cleared after entry into the bloodstream¹ (Galen et al., EcoSal Plus 2016; 7(1)). Therapeutic treatments with the attenuated S. Typhimurium strain VNP20009, while showing great promise in murine models, were unsuccessful despite direct administration of organisms into the bloodstream for several hours (Toso et al., J Clin Oncol 2002; 20(1): 142-52; Heimann et al., J Immunother 2003; 26(2): 179-80). Clinical trials with the licensed S. Typhi vaccine strain Ty21a, expressing the angiogenic TAA VEGFR2 in the resulting carrier vaccine VXM01, also yielded only limited therapeutic benefit, possibly due to over-attenuation of an already greatly weakened parent Ty21a vaccine strain (Niethammer et al., BMC Cancer 2012; 12: 361; Schmitz-Winnenthal et al., Oncoimmunology 2015; 4(4): e1001217; Schmitz-Winnenthal et al., Oncoimmunology 2018; 7(4): e1303584).

Carcinoembryonic antigen (CEA) is a ~180 kDa surface glycoprotein expressed on various tumor tissues including colorectal cancer and hypothesized to have both cell adhesion and pro-angiogenic properties (Hammarstrom et al., Semin Cancer Biol 1999; 9(2): 67-81; Bramswig et al., Cancer Res 2013; 73(22): 6584-96). It is a member of the immunoglobulin gene superfamily comprised of a variable N-terminal domain followed by three sets of constant Ig-like domains, each composed of an “A” and “B” subdomain (designated A1B1, A2B2, and A3B3), and exhibiting over 90% amino acid sequence identity (Hammarstrom et al., Semin Cancer Biol 1999; 9(2): 67-81; Oikawa et al., Biochem Biophys Res Commun 1987; 142(2): 511-8). Multiple cytotoxic T cell epitopes have been mapped to the A3B3 domain for CEA (Hefta et al., Cancer Res 1992; 52(20): 5647-55; Zaremba et al., Cancer Res 1997; 57(20): 4570-7; Nukaya et al., Int J Cancer 1999; 80(1): 92-7). Clinical trials have clearly established the safety of successfully targeting CEA without triggering autoimmunity. Two recent studies targeted dendritic cells with recombinant CEA to expand tumor-specific adaptive immune responses by circumventing both tolerance and circumvent immunosuppression; both trials reported successful induction of clinically relevant CEA-specific T cell and antibody responses (Ullenhag et al., Clin Cancer Res 2004; 10(10): 3273-81.; Morse et al., J Clin Invest 2010; 120(9): 3234-41).

The human mucin gene MUC-1 is another surface expressed and glycosylated colorectal cancer-associated antigen that is also associated with other solid tumors as well. The extracellular protein core of this heterodimeric glycoprotein consists of a variable number of tandem 20 residue proline- and threonine-rich repeat (VNTR) sequences comprising up to 120 copies; the extracellular core is non-covalently associated with an ~20 kDa transmembrane region (Vlad et al., Adv Immunol 2004; 82: 249-93). MUC-1 is normally present on most polarized mucosal epithelial tissue in a heavily glycosylated form. However, when over-expressed in solid tumor tissue, it can become significantly under-glycosylated, which opens up normally masked epitopes to immune surveillance (Cascio et al., Biomolecules 2016; 6(4); von Mensdorff-Pouilly et al., Glycobiology 2005; 15(8): 735-46; Engelmann et al., J Biol Chem 2001; 276(30): 27764-9).

In clinical trials, patients responding to MUC-1 experienced minor side effects, while non-responders were found to have increased levels of circulating myeloid derived suppressor cells potentially interfering with both humoral and cellular tumor-specific immunity (Kimura et al., Cancer Prev Res (Phila) 2013; 6(1): 18-26.; Ma et al., Front Immunol 2019; 10: 1401). A bivalent approach targeting CEA and MUC-1 with a poxviral-based vaccine has recently been confirmed in a Phase 1 dose-escalating clinical trial to be safe and immunogenic (Gatti-Mays et al., Clin Cancer Res 2019; 25(16):4933-44). An immunosuppressive tumor microenvironment (TME) surrounds CRC solid tumors and significantly confounds the ability of the host adaptive immune system to target and eliminate tumor tissue. This microenvironment is comprised of various innate and adaptive immune cells as well as surrounding stromal tissue comprised of cancer associated fibroblasts, vascular tissue, and a rigid extracellular matrix that can exclude infiltrating T cells (Quail et al., Nat Med, (2013), 19:1423-37; Schmitt et al., Nat Rev Immunol, (2021). Tumor-derived cytokines such as TGF-β attenuate the antigen presentation function of dendritic cells, inhibit T cell proliferation and effector function, induce the formation of immunosuppressive T_{reg} cells, and inhibit the function of cytotoxic NK cells (Hanahan et al., Cell, (2011),144:646-74; Nooraei et al., Journal of nanobiotechnology, (2021), 19:59). In addition, tumor-associated macrophages are anergic (M2 polarization), with reduced phagocytic activity and secretion of inflammatory cytokines to promote tumor clearance otherwise carried out by classically active M1 polarized macrophages (Kather et al., Br J Cancer, (2019), 120:871-82; ME et al., Mol Aspects Med, (2019), 69:123-9; Schmitt et al., Nat Rev Immunol, (2021). Given the inaccessibility of tumor tissue and the inherent genetic instability of tumor cells that can rapidly alter surface antigens to evade active immunosurveillance by immune effector cells, the surrounding stromal cell types that are more genetically stable therefore present an attractive alternative therapeutic target for activation of innate immunity, disrupting the immunosuppressive environment of the TME and triggering a tumor-specific adaptive cytotoxic response capable of killing tumor cells; NK cells are particularly attractive for this purpose due to their ability to kill tumor cells without help from T cells (Kather et al., Br J Cancer, (2019), 120:871-82; Quail et al., Nat Med, (2013), 19:1423-37).

Nanomedicine can be defined as any therapeutic modality (nanostructure) having a physical dimension less than 1000 nm (i.e.1 mm) employed for the treatment of disease. In this regard, nanoparticles are typically smaller than ~100 nm (Irvine et al., Nat Rev Immunol, (2020), 20: 321-34). virus-like particles (VLPs) and small liposomes are typically smaller than ~200 nm (Nooraei et al., Journal of nanobiotechnology, (2021), 19:59; Bachmann et al., Nat Rev Immunol, (2010), 10:787-96), and outer membrane vesicles are typically in the range of 30-250 nm (Kulp et al., Annu Rev Microbiol, (2010), 64:163-84; Schwechheimer et al., Nature reviews Microbiology, (2015), 13:605-19). In comparison, viruses are typically smaller than 350 nm, bacteria range in size from 1 - 5 mm, and T cells range from 7 - 20 mm. Recent data from animal models indicates that the intrinsic size and other attendant physical characteristics of nanomedicines alone can facilitate a direct therapeutic effect on tumors, independent of the immunological targeting of tumor-associated antigens, through a phenomenon called enhanced permeation and retention (EPR) that depends on the leaky vasculature of tumors (Irvine et al., Nat Rev Immunol, (2020), 20: 321-34; Fang et al., Advanced drug delivery reviews, (2020), 157:142-60; Kelly et al., Expert review of vaccines, (2019), 18:269-80. The blood vessels of rapidly growing tumors are unlike normal vasculature, having defective endothelial cells, irregular vascular alignment, no smooth muscle layer or innervation, and impaired angiotensin II functional receptors; the resulting leaky tumor vasculature allows for passive extravasation of small nanostructures from the blood into tumor tissue (Fang et al., Advanced drug delivery reviews, (2020), 157:142-60). From within the tumor tissue, these nanostructures can then passively drain through the lymphatic system into regional lymph nodes, potentially encountering and activating antigen presenting cells including dendritic cells (Fang et al., Advanced drug delivery reviews, (2020), 157:142-60; Kelly et al., Expert review of vaccines, (2019), 18:269-80. With larger solid tumors, the vasculature becomes restricted and the EPR effect becomes significantly diminished (Fang et al., Advanced drug delivery reviews, (2020), 157:142-60). This observation may in part explain why the EPR effect has a more therapeutic effect in small animal models than perhaps would be the case in humans; these types of experiments are typically performed on animals with smaller early-stage tumors and leaky vasculature whereas therapeutic treatment of CRC patients typically involves larger late-stage solid tumors which may not respond to EPR as effectively (Fang et al., Advanced drug delivery reviews, (2020), 157:142-60). However, Islam et al. recently reported that coadministration of vasodilators with tumor-targeting nanoparticles significantly improved deposition and regression of very large solid tumors through an enhanced EPR effect in a murine animal model (Islam et al., Journal of personalized medicine, (2021), 11(6). Therefore, nanomedicine offers the possibility of treating “cold” tumors, in which target tumor associated antigen(s) have yet to be identified, through passive targeting and activation of immunosuppressed myeloid cells in tumor tissue through the EPR effect.

It has recently been observed in experimental CRC mouse models that bacterial outer membrane vesicles can also be deposited into murine tumor tissue through the EPR effect, causing a shift in tissue macrophages from anergic M2 to inflammatory M1 cells, infiltration of activated T cells, and associated tumor regression (Kim et al., Nature communications, (2017), 8:626; Qing et al., Advanced materials (Deerfield Beach, Fla), (2020),32: e2002085; Cheng et al., Nature communications, (2021), 12: 2041). Kim at al. tested engineered OMVs from non-pathogenic Gram-negative E. coli W3110 with reduced endotoxicity through deletion of msbB to reduce TLR4 activation and potentially extend the half-life of vesicles in circulation; these vesicles were systemically administered to mice bearing syngeneic subcutaneous tumors from CT26 or MC38 tumor cells (Kim et al., Nature communications, (2017), 8:626). Remarkably, DmsbB OMVs passively accumulated only in tumor tissue, an effect also observed with OMVs isolated from Gram-positive Staphylococcus aureus and Lactobacillus acidophilus. Accumulated vesicles induced IFN-γ responses associated with NK and T cells and elicited full eradication of established tumors with no notable side-effects; interestingly, subcutaneous administration of the host bacteria from which the vesicles were isolated could not induce an anti-tumor response (Kim et al., Nature communications, (2017), 8:626). Qing et al also reported therapeutic deposition of OMVs into tumor tissue but used OMVs coated with calcium phosphate rather than genetic manipulations of the host E coli strain to extend the systemic half-life (Qing et al., Advanced materials (Deerfield Beach, Fla), (2020),32: e2002085). Again, systemically administered vesicles were reported to accumulate in solid CT26-induced tumor tissue, accompanied by a reversal of M1/M2 polarization towards activated M1 macrophages, a significant increase in infiltrating CD8+ T cells, a reduction in immunosuppressive T_{reg}s, an increase in tumor apoptotic cells, and higher overall survival rates (Qing et al., Advanced materials (Deerfield Beach, Fla), (2020),32: e2002085). In a further advancement of this OMV therapeutic strategy, Cheng et al. used recombinant systemically administered rOMVs, engineered in E. coli to target the Adpgk neoantigen present in MC38 colon cancer cells, to once again target established solid tumors (Cheng et al., Nature communications, (2021), 12: 2041). Treatment led to complete regression of tumors in 60% of mice, with CD4+ and CD8+ T cells, activated neutrophils, and DCs all significantly elevated in MC38 tumor tissues after subcutaneous immunization, but reduced levels of immunosuppressive T_{reg}s (Cheng et al., Nature communications, (2021), 12: 2041).

There is an urgent need to develop new compositions and methods for treating cancer. The present invention satisfies this need and provides additional advantages as well.

This background information is provided for informational purposes only. No admission is necessarily intended, nor should it be construed, that any of the preceding information constitutes prior art against the present invention.

### SUMMARY OF THE INVENTION

It is to be understood that both the foregoing general description of the embodiments and the following detailed description are exemplary, and thus do not restrict the scope of the embodiments.

In one aspect, the invention provides a live Salmonella Typhi vector that has been engineered to express one or more cancer antigens; an outer membrane folding protein BamA or a fragment or variant thereof; and a lipid A deacylase PagL or a fragment or variant thereof, wherein the Salmonella Typhi vector is capable of delivering the antigen to a mucosal tissue via an outer membrane vesicle when administered to a subject.

In some embodiments, expression of one or more of the BamA, PagL and antigen is inducible and under the control of an inducible promoter. In some embodiments, the promoter is sensitive to osmolarity. In some embodiments, the osmotically controlled inducible promoter is a promoter of Outer Membrane Protein C (ompC) gene. In some embodiments, the antigen comprises an outer membrane protein, an antigenic fragment thereof or a variant thereof.

In some embodiments, the cancer antigen is from colon cancer. In some embodiments, the invention provides an attenuated S. Typhi-bacterial live vector vaccine strain expressing a fusion protein comprising antigenic sequences from the proteins CEA and MUC1, wherein the S. Typhi-bacterial live vector exhibits enhanced delivery of the fusion protein to the immune system through increased formation of recombinant outer membrane vesicles (rOMVs). In some embodiments, the S. Typhi-bacterial live vector expresses a ClyA protein that is exported from the live vaccine via rOMVs. In some embodiments, there is increased extracellular export of the fusion protein and ClyA from the live vaccine via rOMVs.

In another aspect, the invention provides a composition comprising isolated recombinant outer membrane vesicles from Salmonella Typhi comprising one or more cancer antigens, wherein the Salmonella Typhi has been engineered to express the antigen.

In another aspect, the invention provides a method of inducing an immune response in a subject in need thereof, comprising administering to the subject an immunologically-effective amount of a live Salmonella enterica Typhi vector that has been engineered to express one or more cancer antigens; an outer membrane folding protein BamA or a fragment or variant thereof; and a lipid A deacylase PagL or a fragment or variant thereof, wherein the Salmonella Typhi vector is capable of delivering the antigen to a mucosal tissue via an outer membrane vesicle when administered to a subject.

In another aspect, the invention provides a method of inducing an immune response in a subject in need thereof, comprising administering to the subject an immunologically-effective amount of isolated recombinant outer membrane vesicles from a live Salmonella Typhi vector comprising one or more cancer antigens; wherein the Salmonella Typhi vector has been engineered to express one or more cancer antigens; an outer membrane folding protein BamA or a fragment or variant thereof; and a lipid A deacylase PagL or a fragment or variant thereof.

In another aspect, the invention provides a live Salmonella Typhi vector that has been engineered to express:
a. one or more cancer antigens; and
b. a lipid A deacylase PagL or a fragment or variant thereof,
wherein the Salmonella Typhi vector is capable of delivering the antigen to a mucosal tissue via an outer membrane vesicle when administered to a subject. In another aspect, the invention provides a composition comprising isolated recombinant outer membrane vesicles comprising one or more cancer antigens from the Salmonella Typhi vector.

In another aspect, the invention provides a method of inducing an immune response in a subject in need thereof, comprising administering to the subject an immunologically-effective amount of a live Salmonella Typhi vector that has been engineered to express
a. one or more cancer antigens; and
b. a lipid A deacylase PagL or a fragment or variant thereof,
wherein the antigen is delivered to a mucosal tissue of the subject by an outer membrane vesicle produced by the Salmonella Typhi vector.

In another aspect, the invention provides a method of inducing an immune response in a subject in need thereof, comprising administering to the subject
i. an immunologically-effective amount of a live Salmonella Typhi vector that has been engineered to express:
   a. one or more cancer antigens; and
   b. a lipid A deacylase PagL or a fragment or variant thereof; and
iii. an immunologically-effective amount of isolated recombinant outer membrane vesicles from a live Salmonella Typhi vector, wherein the Salmonella Typhi vector has been engineered to express the one or more cancer antigens; an outer membrane folding protein BamA or a fragment or variant thereof; and a lipid A deacylase PagL or a fragment or variant thereof.

In another aspect, the invention provides a method of inducing an immune response in a subject in need thereof, comprising administering to the subject an immunologically-effective amount of a live Salmonella Typhi vector that has been engineered to express
a. one or more cancer antigens; and
b. a lipid A deacylase PagL or a fragment or variant thereof,
wherein the antigen is delivered to a mucosal tissue of the subject following early detection of the targeted tumor by unrelated diagnostic methodologies. See, e.g., Cohen et al. 2018. Science, 359: 926-930.

In another aspect, the invention provides a method of treating or preventing cancer in a subject, comprising administering to the subject an effective amount of a live Salmonella Typhi vector as described herein and/or an effective amount of isolated recombinant outer membrane vesicles as described herein.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The skilled artisan will understand that the drawings, described below, are for illustration purposes only. The drawings are not intended to limit the scope of the present teachings in any way.

FIG. 1. Hemolytic activity of isogenic attenuated S. Typhi CVD 910 live vector strains expressing chromosomally encoded ClyA exported by over-expression of PagL. Samples from approximately 2 × 10⁷ CFU of synchronized bacterial cultures were analyzed for hemolytic activity using sheep red blood cells, with five measurements per group. Lane 1: PBS; Lane 2: 910; Lane 3: 910ΔguaBA::clyA; Lane 4: 910AguaBA::clyA(pPagL).

FIG. 2. Export of antigen OmpA^{Ab} in OMVs from CVD 910 live vaccine strains.

FIG. 3. Hemolytic activity of isogenic attenuated S Typhi CVD 910 live vector strains expressing chromosomally encoded ClyA exported by over-expression of PagL. Samples from approximately 2 × 10⁷ CFU of synchronized bacterial cultures were analyzed for hemolytic activity using sheep red blood cells, with five measurements per group. Lane 1: PBS; Lane 2: 910; Lane 3: 910ΔguaBA::clyA; Lane 4: 910ΔguaBA::clyA(pPagLv1); Lane 5: 910ΔguaBA::clyA(pPagLv2); Lane 6: 910ΔguaBA::clyA(pPagLv3).

FIG. 4. Hemolytic activity of isogenic attenuated S Typhi CVD 910 live vector strains expressing chromosomally encoded ClyA exported by over-expression of BamA. Samples from approximately 2 × 10⁷ CFU of synchronized bacterial cultures were analyzed for hemolytic activity using sheep red blood cells, with five measurements per group. Lane 1: PBS; Lane 2: 910; Lane 3: 910(pSEC10); Lane 4: 910ΔguaBA::clyA; Lane 5: 910ΔguaBA::clyA(pAbBamAv1); Lane 6: 910ΔguaBA::clyA(pAbBamAv2).

FIG. 5. Candidate bivalent S. Typhi-based colorectal immunotherapeutic vaccine. Targeted colorectal antigen domains from carcinoembryonic antigen (CEA) and the human mucin MUC-1 is expressed as a surface-expressed fusion protein, subsequently presented to immune inductive sites via a novel inducible PagL-mediated outer membrane vesicle (OMV) delivery system. Plasmid is genetically stabilized using a novel single stranded binding protein-based strategy.

FIG. 6. Targeted colorectal cancer-associated antigens to be expressed in an attenuated S. Typhi-based bivalent carrier vaccine. A. Carinoembryonic Antigen (CEA). B. MUC-1.

FIG. 7. CRC Cancer Antigen(s) Cassette.

FIG. 8. Engineered modifications to lipid A structure to reduce TLR 4 activation. Schematic representation of Salmonella lipid A structure with cleavage sites indicated for PagL and LpxE.

FIG. 9. Genetic structure of CVD 910ΔguaBA::P_{ompC}-bamA^{Ab}ΔƒliC::P_{ompC}lpxEFn(pPagL-LOAM). Graphic depicts the chromosomal insertion of P_{ompC}-lpxEFn to replace the ƒliC gene while preserving the original upstream P_{ƒliC} promoter, thereby creating a nested set of P_{ƒliC}-P_{ompC} promoters; this same strategy was also employed with the chromosomal insertion of P_{ompC}-bamA^{Ab} to replace the guaBA genes while preserving the original upstream P_{guaBA} promoter, thereby creating a nested set of P_{guaBA}-P_{ompC} promoters.

FIG. 10. In vitro characterization of CRC fusion protein expression in purified rOMVs. 0.5 µg of purified rOMVs were loaded per lane in an SDS-PAGE gel stained with Coomassie Brilliant Blue (Panel A) and further characterized by western immunoblot analysis using purified rabbit A3B3-specifc primary antibody and Alexa Fluor 680 goat anti-rabbit IgG (H+L) secondary antibody.

FIG. 11. IFN-γ responses against A3B3-MUC1 in BALB/c mice immunized intramuscularly with 2 µg of rOMVs on days 0 and 21. Splenocytes were harvested on day 35, pooled for each group, and immediately used for ELISpot analysis without frozen storage. Each pooled sample was analyzed in quadruplicate; cells were stimulated with 10 mg/ml of A3B3-MUC1 purified antigen for 40 hours prior to score spots; 250,000 splenocytes were analyzed per well.

FIG. 12. IFN-γ responses against A3B3-MUC1 in C57BL/6 mice immunized as described in Table 4. Splenocytes were harvested on day 21, pooled for each group, and immediately used for ELISpot analysis without frozen storage. See text for further experimental details.

FIG. 13. Immunotherapeutic treatment of C57BL/6 mice implanted with 300,000 MC38 colon cancer cells engineered for constitutive expression of either MUC1 or CEAv2 antigen; CEAv2 is a truncated version of CEA homologous to the domain expressed by our rOMVs. (A and B). Cells were implanted subcutaneously on day 0; mice were then treated on days 3, 5, 7, and 9 (red arrows) with intravenous injections of either with PBS or 0.75 mg per dose of either ΔƒliC or lpxE rOMVs expressing a CEA-MUC1 targeted tumor-associated fusion protein. Progression of tumors as measured by tumor volume are plotted as mean volumes with standard error of the means bars. Statistical significance on day 28 was evaluated by ANOVA with Geisser-Greenhouse correction and a Tukey multiple comparisons analysis. For mice implanted with MC38-MUC1, ΔƒliC vs. lpxE, p = 0.0483; ΔƒliC vs. PBS, p = 0.0001; lpxE vs. PBS p=0.0004; for mice implanted with MC38-CEAv2, ΔƒliC vs. IpxE, p = 0.2202; ΔƒliC vs. PBS, p = 0.2122; lpxE vs. PBS p=0.0178. (C and D). Cells were implanted subcutaneously and allowed to grow to a mean tumor volume of 115 mm³ for CEAv2 and 120 mm³ for MUC1; mice were then treated on days 0, 2, 4, and 6 (red arrows) with intravenous injections of either PBS or 0.75 µg per dose of rOMVs. Tumor progression, plots, and statistical analysis are as presented in panels A and B. CEAv2-lpxE vs. MUC1-lpxE is not statistically significant, nor is CEAv2-ΔƒliC vs. MUC1-DfliC. (E.) Kaplan-Meier survival curves for experiment plotted in Panels C and D. Differences in survival of CEAv2-ΔƒliC vs. MUC1-ΔƒliC and MUC1-ΔƒliC vs. MUC1-lpxE are not significant.

FIG. 14. Immunogenicity and therapeutic efficacy of re-engineered rOMVs expressing individual domains of the CRC fusion protein, compared to the un-modified rOMV expressing full-length A3B3-MUC1 fusion protein. A: graphic describing the reengineering strategy for construction of rOMVs expressing single CRC domains. B: anti-A3B3-MUC1 serum IgG titers. C: ELISPOT assay measuring IFN-γ secretion of isolated splenocytes stimulated with A3B3-MUC1 fusion protein. D: Tumor volumes measured in C57BL/6 mice challenged on day 0 with MC38-CEAv2 and treated intravenously on days 3, 5, 7, and 9 with 0.75 mg of rA3B3^{ΔƒliC}, rMUC1 ^{ΔƒliC}, rOMV^{ΔƒliC-CRC,} or empty rOMVA^{ΔƒliC-} ^{pagL} vesicles.

### DETAILED DESCRIPTION

Reference will now be made in detail to the presently preferred embodiments of the invention which, together with the drawings and the following examples, serve to explain the principles of the invention. These embodiments are described in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

The practice of the present invention employs, unless otherwise indicated, conventional techniques of molecular biology (including recombinant techniques), microbiology, cell biology, biochemistry and immunology, which are within the skill of the art. Such techniques are explained fully in the literature. See, e.g., Sambrook et al. Molecular Cloning: A Laboratory Manual, 2^{nd} edition (1989); Current Protocols in Molecular Biology (F. M. Ausubel et al. eds. (1987)); the series Methods in Enzymology (Academic Press, Inc.); PCR: A Practical Approach (M. MacPherson et al. IRL Press at Oxford University Press (1991)); PCR 2: A Practical Approach (M. J. MacPherson, B. D. Hames and G. R. Taylor eds. (1995)); Antibodies, A Laboratory Manual (Harlow and Lane eds. (1988)); Using Antibodies, A Laboratory Manual (Harlow and Lane eds. (1999)); and Animal Cell Culture (R. I. Freshney ed. (1987)).

Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341).

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” means plus or minus 10% of the numerical value of the number with which it is being used.

### Live Vectors and Recombinant Outer Membrane Vesicles

In some embodiments, the invention provides a live Salmonella vector, such as S. Typhi, wherein the Salmonella vector has been engineered to express one or more cancer antigens; an outer membrane folding protein BamA or a fragment or variant thereof; and a lipid A deacylase PagL or a fragment or variant thereof, wherein the Salmonella vector is capable of delivering the antigen to a mucosal tissue via an outer membrane vesicle when administered to a subject.

In some embodiments, the invention provides a live Salmonella Typhi vector that has been engineered to express one or more cancer antigens and a lipid A deacylase PagL or a fragment or variant thereof, wherein the Salmonella Typhi vector is capable of delivering the antigen to a mucosal tissue via an outer membrane vesicle when administered to a subject.

BamA is an ~90 kDa protein that constitutes an essential component of a 5-protein outer membrane β-barrel assembly machinery (BAM) complex that catalyzes the insertion of β-barrel proteins into the outer membrane of Gram negative bacteria. The BamA which can be used in the invention is not particularly limiting. BamA encompasses full length BamA as well as biologically active fragments and variants of BamA. In some embodiments, BamA is from Acinetobacter baumannii. In some embodiments, the nucleotide sequence comprising BamA has been optimized. In some embodiments, one or more codons (e.g., rare codons) have been optimized to enhance expression. In some embodiments, the putative ribosome binding sites have been optimized to enhance expression. In some embodiments, the amino acid sequence of BamA is SEQ ID NO:8. In some embodiments, the nucleic acid sequence of BamA is SEQ ID NO:10.

The lipid A deacylase PagL which can be used in the invention is not particularly limiting. PagL encompasses full length PagL as well as biologically active fragments and variants of PagL. In some embodiments, PagL is from Salmonella enterica. In some embodiments, PagL is from the Salmonella enterica serovar Typhimurium. In some embodiments, the nucleotide sequence comprising PagL has been optimized. In some embodiments, one or more codons (e.g., rare codons) have been optimized to enhance expression. In some embodiments, the putative ribosome binding sites have been optimized to enhance expression. In some embodiments, the nucleotide sequence of PagL comprises SEQ ID NOS: 1, 3 or 5. In some embodiments, the amino acid sequence of PagL comprises SEQ ID NOS:2 or 4.

In some embodiments, the S. Typhi-bacterial live vector over-expresses one or more further vesicle-catalyzing proteins such as ClyA responsible for naturally inducing OMV formation in S. Typhi. ClyA encompasses full length ClyA as well as biologically active fragments and variants of ClyA.

ClyA is an endogenous protein in S. Typhi, that can catalyze the formation of large outer membrane vesicles when overexpressed. Such a mechanism for vesicle formation raised the intriguing possibility of engineering ClyA to export from a live vector, via vesicles, heterologous foreign antigens; these vesicles could also carry immunomodulatory lipopolysaccharide (LPS) to perhaps improve the immunogenicity of an otherwise poorly immunogenic antigen. The utility of ClyA for enhancing the immunogenicity of the foreign Protective Antigen (PA83) from anthrax toxin, a strategy which produced a live vector anthrax vaccine proven to be immunogenic in both mouse and non-human primate animal models (Galen et al. 2010. Infect. Immun. 78(1):337-47; Galen et al. 2009. J. Infect. Dis. 199(3):326-35) has been confirmed. Like ClyA, over-expression of PagL has also been recently reported to induce prolific formation of outer membrane vesicles (Elhenawy et al. 2016. mBio. 7(4):e00940-16. doi: 10.1128/mBio.00940-16) interestingly, although the pagL gene is present in the murine pathogen S. Typhimurium, it is absent in S. Typhi.

ClyA from S. Typhi was first described by Wallace et al., who also reported the crystal structure for the homologous HlyE hemolysin from E. coli. (Wallace et al., 2000. Cell 100:265-276.). ClyA protein can cause hemolysis in target cells. The present invention encompasses use of both hemolytically active and hemolytically inactive forms of ClyA, with hemolytically inactive mutant forms being more preferred where preservation of antigen export and immunogenicity of the resulting proteins can be maintained. In some embodiments, the nucleotide and amino acid sequence of ClyA corresponds to SEQ ID NOS: 15 and 16, respectively. In some embodiments, the ClyA is mutated to reduce the hemolytic activity of ClyA while still retaining the export function of ClyA. In one embodiment, the ClyA mutant is ClyA I198N. In another embodiment, the ClyA mutant is ClyA C285W. In some embodiments, the ClyA is mutated to reduce hemolytic activity of ClyA. In some embodiments, the ClyA mutant is selected from the group consisting of ClyA I198N, ClyA C285W, ClyA A199D, ClyA E204K. In some embodiments, the ClyA is a fusion protein. In some embodiments, the ClyA comprises I198N, A199D, and E204K substitution mutations. The mutant sequences are with reference to SEQ ID NO:16.

In some embodiments, the Salmonella Typhi vector has been engineered to reduce both TLR4- and TLR5-mediated reactogenicity. In some embodiments, the Salmonella Typhi vector has a deletion in the fliC gene which is a TLR5 agonist. In some embodiments, the sequence of the fliC gene to be deleted from the chromosome of a candidate attenuated S. Typhi vaccine strain such as CVD910, or derivatives thereof, is SEQ ID NO:29.

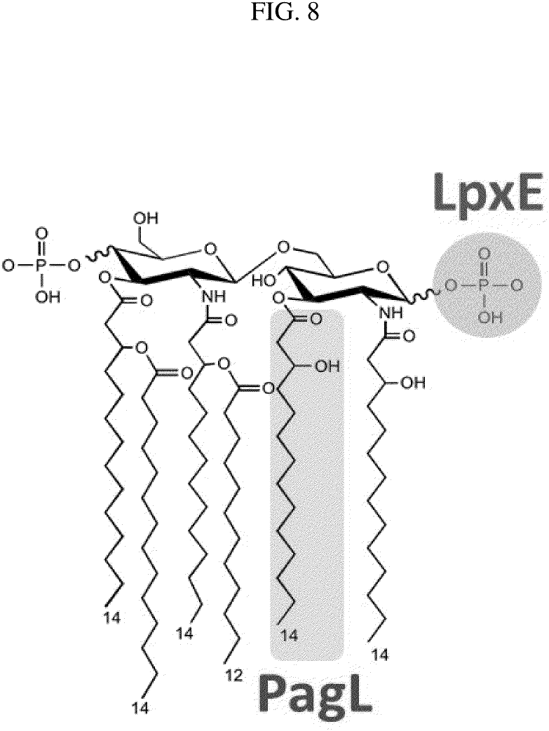

In some embodiments, the Salmonella Typhi vector has been engineered to express lpxE from Francisella novicida (lpxE^{Fn} ). In some embodiments, the nucleotide sequence of lpxE is SEQ ID NO:26 and the amino acid sequence is SEQ ID NO:27. LpxE is a lipid A 1-phosphatase which dephosphorylates lipid A to produce a less reactogenic monophosphoryl species (FIG. 8). Through co-expression of PagL (which deacylates lipid A while promoting hypervesiculation) rOMVs can be produced containing pentaacylmonophosphoryl-lipid A with significantly reduced TLR 4 and TLR5 activity. In some embodiments, the Salmonella Typhi vector is engineered to insert nucleic acid sequence encoding LpxE into the fliC locus of S. Typhi.

In some embodiments, the invention provides a composition comprising isolated recombinant outer membrane vesicles from the Salmonella Typhi vectors of the invention comprising one or more cancer antigens. In some embodiments, the antigen comprises an outer membrane protein, an antigenic fragment thereof or a variant thereof, wherein the Salmonella Typhi has been engineered to express the antigen.

In some embodiments, the cancer is selected from colon, colorectal, leukemia (e.g., chronic lymphocytic leukemia or acute myeloid leukemia) lymphoma (e.g., Non-Hodgkin Lymphoma), breast, prostate, liver, pancreatic, brain, lung (e.g., small cell or non-small cell lung cancer) and skin cancer (e.g., melanoma), uterine, gallbladder, adenocarcinoma, cholangiocarcinoma, esophageal, gastric, glioblastoma, ovarian, urinary bladder cancer, and head and neck cancer. In some embodiments, the cancer is colon cancer.

In some embodiments, the cancer antigen comprises one or more of the following antigens (or antigenic fragments or derivatives) selected from neo-antigen, carcinoembryonic antigen (CEA), human epithelial mucin MUC-1, the cancer-testis antigen NY-ESO-1, HER2/neu, SART-1, SART-2, KIAA0156, ART-1, ART-4, cyclophilin B, mutated elongation factor 2, malic enzyme, and alpha-actinin-4, eIF4G, aldolase, annexin XI, Rip-1, and NY-LU-12, fibromodulin, RHAMM/CD168, MDM2, hTERT, the oncofetal antigen-immature laminin receptor protein (OFAiLRP), adipophilin, survivin, KW1 to KW14 and the tumor-derived IgVHCDR3 region, RHAMM-derived R3 peptide, B7.1, ICAM-1, LFA-3, epidermal growth factor receptor variant III (EGFR_{V}III), heat shock protein, IL-13 receptor alpha 2, alpha fetoprotein, MART1, gp100, cancer-testis antigen MAGEA3, L-BLP25, p53, Wilms tumor-1, KRAS, reactive telomerase, gastrin, prostate-specific antigen (PSA), tumor-associated antigen 5T4, cancer-testis antigens MAGE, PASD1, the B-cell antigen CD20 and viral oncoproteins E6 or E7.

In some embodiments, the cancer antigen comprises one or more antigenic fragments of CEA and/or MUC1. In some embodiments, the CEA antigen and MUC1 antigen are part of a fusion protein. In some embodiments, the CEA antigen and MUC1 antigen are part of the same fusion protein.

In some embodiments, the CEA antigen comprises domain A3B3. In some embodiments, the amino acid sequence of domain A3B3 comprises SEQ ID NO:23. In some embodiments, the nucleotide sequence of domain A3B3 comprises SEQ ID NO:30.

In some embodiments, the MUC1 antigen is a fragment comprising multiple repeat domains. In some embodiments, the amino acid sequence of the MUC1 fragment comprises SEQ ID NO:24. In some embodiments, the nucleotide sequence of the MUC1 fragment comprises SEQ ID NO:31.

In some embodiments, the cancer antigen is expressed in S. Typhi as a fusion protein. In some embodiments, the cancer antigen is fused to a polypeptide sequence comprising a surface presentation protein. In some embodiments, the polypeptide sequence comprising a surface presentation protein is selected from Lpp-OmpA, Lpp-OmpT and ClyA. In some embodiments, the amino acid sequence of Lpp-OmpA comprises SEQ ID NO:21. In some embodiments, the nucleotide sequence of lpp-ompA comprises SEQ ID NO:28.

In some embodiments, the invention provides an isolated nucleic acid encoding an expression cassette for expression in the S. Typhi vectors of the invention, wherein the expression cassette encodes a fusion protein comprising a surface presentation protein and one or more antigens. In some embodiments, the surface expression protein is selected from Lpp-OmpA, Lpp-OmpT and ClyA.

In some embodiments, the cancer antigen is a fusion protein comprising CEA domain A3B3 and a MUC1 fragment fused to a surface presentation protein such as Lpp-OmpA. In some embodiments, the cancer antigen comprises a Lpp-OmpA:A3B3:MUC1 fusion protein comprising the amino acid sequence of SEQ ID NO:12. In some embodiments, the cancer antigen comprises a lpp-ompA:a3b3:muc1 gene fusion encoded by SEQ ID NO:11.

In some embodiments, the cancer antigen is a fusion protein comprising CEA domain A3B3 fused to a surface presentation protein such as Lpp-OmpA. In some embodiments, the cancer antigen comprises a Lpp-OmpA:A3B3 fusion protein comprising the amino acid sequence of SEQ ID NO:14. In some embodiments, the cancer antigen comprises a Lpp-OmpA:A3B3 gene fusion encoded by SEQ ID NO:13.

In some embodiments, the cancer antigen is a fusion protein comprising a MUC1 fragment fused to a surface presentation protein such as Lpp-OmpA. In some embodiments, the cancer antigen comprises a Lpp-OmpA:MUC1 fusion protein comprising the amino acid sequence of SEQ ID NO:18. In some embodiments, the cancer antigen comprises a lpp-ompA:muc1 gene fusion protein encoded by SEQ ID NO:17.

In some embodiments, the cancer antigen comprises a ClyA^{I198N}:A3B3:MUC1 fusion protein comprising the amino acid sequence of SEQ ID NO:20. In some embodiments, the cancer antigen comprises a clyA^{I198N}:a3b3:muc1 fusion protein encoded by SEQ ID NO:19. In some embodiments, the ClyA^{I198N} amino acid sequence comprises SEQ ID NO:25.

In some embodiments, the polypeptide components of the fusion protein are separated by one or more linker amino acid sequences. In some embodiments, the linker amino acid sequence is SEQ ID NO:22. In preferred embodiments, the cancer antigen exhibits no glycosylation.

In some embodiments, the invention provides a nucleic acid comprising any of SEQ ID NOS:11, 13, 17, or 19.

In some embodiments, the invention provides a composition comprising a combination of isolated recombinant outer membrane vesicles from the engineered Salmonella Typhi vectors as described herein.

In some embodiments, the invention provides genetically engineered attenuated strains of S. Typhi as live vaccine platforms for delivery of one or more cancer antigens to protect against the development and/or progression of cancer such as colon cancer. The one or more antigens can be expressed on the surface of live vaccines after induction of synthesis in vivo, and will be exported from the surface to immune inductive sites via a unique inducible OMV-mediated export system, as described in more detail below.

In some embodiments, the live vaccines will express the cancer antigens carcinoembryonic antigen (CEA) and human epithelial mucin MUC-1 and be useful as a colon cancer vaccine.

The Salmonella Typhi strain that can be used in the present invention as a vaccine is not limiting. For example, it can include any particular strain that has been genetically attenuated from the original clinical isolate Ty2. Any attenuated Salmonella Typhi strain derived from Ty2 can be used as a live vector in accordance with the invention. Non-limiting, exemplary attenuated Salmonella Typhi strains include S. Typhi Ty21a, CVD 908, S. Typhi CVD 909, CVD 908-htrA, CVD 915, and CVD 910. In some embodiments, the S. Typhi strain can carry one or more additional chromosomal mutations in an essential gene that is expressed on a plasmid. In some embodiments, the plasmid also encodes a heterologous protein in accordance with the invention, enabling selection and genetic stabilization of the plasmid and preventing loss in S. Typhi. In some embodiments, the S. Typhi strain carries a mutation in the ssb gene which is encoded on a selection expression plasmid.

If heterologous antigens or other proteins are overexpressed using plasmids, plasmid stability can be a key factor in the development of high quality attenuated S. Typhi vaccines. Plasmidless bacterial cells tend to accumulate more rapidly than plasmid-bearing cells. One reason for this increased rate of accumulation is that the transcription and translation of plasmid genes imposes a metabolic burden which slows cell growth and gives plasmidless cells a competitive advantage. Furthermore, foreign plasmid gene products are sometimes toxic to the host cell. Thus, it is advantageous for the plasmid to be under some form of selective pressure, in order to ensure that the encoded antigens are properly and efficiently expressed, so that a robust and effective immune response can be achieved.

In some embodiments, the plasmid is selected within S. Typhi using a non-antibiotic selection system. For example, the plasmid can encode an essential gene that complements an otherwise lethal deletion/mutation of this locus from the live vector chromosome. Exemplary non-antibiotic expression plasmids that can be used in the invention are described herein and further plasmid systems which can be used in the invention are described, for example, in U.S. Patent Appl. Pub. No. 2007/0281348, U.S. Pat. Nos. 7,141,408, 7,138,112, 7,125,720, 6,977,176, 6,969,513, 6,703,233, and 6,413,768, which are herein incorporated by reference.

In one embodiment, a non-antibiotic genetic stabilization and selection system for expression plasmids is engineered to encode single-stranded binding protein (SSB), an essential protein involved in DNA replication, recombination, and repair which can be deleted from the S. Typhi live vector chromosome (Lohman et al., Annu Rev Biochem. 1994; 63:527-570; Chase et al., Annu Rev Biochem. 1986; 55:103-136; Galen et al., Infect Immun. 2010 January; 78(1):337-47). In some embodiments, the plasmid expression vector for S. Typhi encodes a single-stranded binding protein (SSB). In some embodiments, the expression vector is pSEC10S.

In some embodiments of the invention, expression plasmids are employed in which both the random segregation and catalytic limitations inherent in non-antibiotic plasmid selection systems have been removed. The segregation of these plasmids within S. Typhi live vectors is improved using an active partitioning system (parA) for S. Typhi CVD 908-htrA (Galen et al., Infect. Immun. 67:6424-6433). In some embodiments, dependence on catalytic enzymes is avoided by using a plasmid selection/post-segregational killing system based on the ssb gene.

A solution to the instability of multicopy plasmids and the foreign antigens they encode is to integrate foreign gene cassettes into the chromosome of the live vector. However, the drop in copy number becomes both an advantage and a disadvantage; while the reduced copy number will certainly reduce the metabolic burden associated with both the multicopy plasmid itself and the encoded foreign protein(s), this reduction in foreign antigen synthesis ultimately leads to reduced delivery of these antigens to the host immune system and possibly reduced immunogenicity. This explanation could account for why in clinical trials serum immune responses to chromosomally encoded antigens have to date been modest. (Gonzalez et al., J Infect Dis. 1994; 169:927-931; Khan et al., Vaccine 2007; 25:4175-4182).

An antigenic or biologically active fragment is a polypeptide having an amino acid sequence that entirely is the same as part but not all of the amino acid sequence of one of the polypeptides. The antigenic fragment can be “free-standing,” or comprised within a larger polypeptide of which they form a part or region, most preferably as a single continuous region.

In some embodiments, the antigenic or biologically active fragments include, for example, truncation polypeptides having the amino acid sequence of the polypeptides, except for deletion of a continuous series of residues that includes the amino terminus, or a continuous series of residues that includes the carboxyl terminus or deletion of two continuous series of residues, one including the amino terminus and one including the carboxyl terminus. In some embodiments, fragments are characterized by structural or functional attributes such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, and high antigenic index regions.

The fragment can be of any size. An antigenic fragment is capable of inducing an immune response in a subject or be recognized by a specific antibody. In some embodiments, the fragment corresponds to an amino-terminal truncation mutant. In some embodiments, the number of amino terminal amino acids missing from the fragment ranges from 1-100 amino acids. In some embodiments, it ranges from 1-75 amino acids, 1-50 amino acids, 1-40 amino acids, 1-30 amino acids, 1-25 amino acids, 1-20 amino acids, 1-15 amino acids, 1-10 amino acids and 1-5 amino acids.

In some embodiments, the fragment corresponds to carboxyl-terminal truncation mutant. In some embodiments, the number of carboxyl terminal amino acids missing from the fragment ranges from 1-100 amino acids. In some embodiments, it ranges from 1-75 amino acids, 1-50 amino acids, 1-40 amino acids, 1-30 amino acids, 1-25 amino acids, 1-20 amino acids, 1-15 amino acids, 1-10 amino acids and 1-5 amino acids.

In some embodiments, the fragment corresponds to an internal fragment that lacks both the amino and carboxyl terminal amino acids. In some embodiments, the fragment is 7-200 amino acid residues in length. In some embodiments, the fragment is 10-100 amino acid residues, 15-85 amino acid residues, 25-65 amino acid residues or 30-50 amino acid residues in length. In some embodiments, the fragment is 7 amino acids, 10 amino acids, 12 amino acids, 15 amino acids, 20 amino acids, 25 amino acids, 30 amino acids, 35 amino acids, 40 amino acids, 45 amino acids, 50 amino acids 55 amino acids, 60 amino acids, 80 amino acids or 100 amino acids in length.

In some embodiments, the fragment is at least 50 amino acids, 100 amino acids, 150 amino acids, 200 amino acids or at least 250 amino acids in length. Of course, larger antigenic fragments are also useful according to the present invention, as are fragments corresponding to most, if not all, of the amino acid sequence of the polypeptides described herein.

In some embodiments, the invention provides a polypeptide comprising any of SEQ ID NOS:12, 14, 18, or 20.

In some embodiments, the polypeptides have an amino acid sequence at least 80, 85%, 90%, 91%, 92%, 93%, 94%, 95%, 96%, 97%, 98%, 99%, or 100% identical to the polypeptides described herein or antigenic or biologically active fragments thereof. In some embodiments, the variants are those that vary from the reference by conservative amino acid substitutions, i.e., those that substitute a residue with another of like characteristics. Typical substitutions are among Ala, Val, Leu and Ile; among Ser and Thr; among the acidic residues Asp and Glu; among Asn and Gln; and among the basic residues Lys and Arg, or aromatic residues Phe and Tyr. In some embodiments, the polypeptides are variants in which several, 5 to 10, 1 to 5, or 1 to 2 amino acids are substituted, deleted, or added in any combination.

In some embodiments, the polypeptides are encoded by polynucleotides that are optimized for high level expression in Salmonella using codons that are preferred in Salmonella. As used herein, a codon that is “optimized for high level expression in Salmonella” refers to a codon that is relatively more abundant in Salmonella in comparison with all other codons corresponding to the same amino acid. In some embodiments, at least 10% of the codons are optimized for high level expression in Salmonella. In some embodiments, at least 25%, at least 50%, at least 75%, at least 80%, at least 85%, at least 90%, at least 95%, or at least 99% of the codons are optimized for high level expression in Salmonella.

In some embodiments, the cancer antigen is expressed on a plasmid in S. Typhi In some embodiments, the plasmid has a non-antibiotic based plasmid selection and genetic stabilization system. In some embodiments, the plasmid expresses a gene that is essential for the growth of S. Typhi and has been chromosomally mutated in S. Typhi. In some embodiments, the gene encodes single stranded binding protein (SSB).

In some embodiments, the cancer antigen is chromosomally integrated in S. Typhi. It will be appreciated that inserting the gene cassette(s) into, e.g., the guaBA, htrA, ssb, and/or rpoS locus of S. Typhi can be accomplished, for example, using the lambda Red recombination system (Datsenko et al., PNAS. 2000. 97(12): 6640-5). In some embodiments, the cancer antigen is inserted into the guaBA locus of S. Typhi. In some embodiments, the cancer antigen is inserted into the rpoS locus of S. Typhi.

In some embodiments, immunogenic cassettes can be integrated into either the ΔguaBA or ΔrpoS locus of CVD 910ssb, for example, to compare the immunogenicity of chromosomal integrations versus antigen-specific immunogenicity elicited by plasmid-based expression. In some embodiments, only the open reading frames of ΔguaBA and ΔrpoS are deleted, leaving the original promoters for these sites intact. In some embodiments, insertion cassettes include the P_{ompC} promoter from the low copy expression plasmids, such that integration into ΔguaBA or ΔrpoS results in nested promoters controlling inducible expression of a given cassette at two levels.

### Pharmaceutical Compositions

In some embodiments, the invention provides pharmaceutical compositions comprising S. Typhi live vector vaccines of the invention. Such compositions can be for use in vaccination of individuals, such as humans. Such pharmaceutical compositions may include pharmaceutically effective carriers, and optionally, may include other therapeutic ingredients, such as various adjuvants known in the art. Non-limiting examples of pharmaceutically acceptable carriers or excipients include, without limitation, any of the standard pharmaceutical carriers or excipients such as phosphate buffered saline solutions, water, emulsions such as oil/water emulsions, microemulsions, and the like.

In some embodiments, the composition comprises one or more live S. Typhi live vectors of the invention. In some embodiments, the composition comprises a combination of live Salmonella Typhi vectors.

In some embodiments, the invention provides a composition comprising isolated recombinant outer membrane vesicles from a live Salmonella Typhi vector of the invention, comprising one or more cancer antigens expressed from the Salmonella Typhi vector.

In some embodiments, the invention provides a composition comprising a combination of isolated recombinant outer membrane vesicles from live Salmonella Typhi vectors of the disclosure.

In some embodiments, the cancer antigen is an antigenic fragment of CEA, MUC1 or both. In some embodiments, the cancer antigen comprises a fusion protein comprising lpp-ompA or lpp-ompT and antigenic fragments of CEA and MUC1.

The carrier or carriers must be pharmaceutically acceptable in the sense that they are compatible with the therapeutic ingredients and are not unduly deleterious to the recipient thereof. The therapeutic ingredient or ingredients are provided in an amount and frequency necessary to achieve the desired immunological effect.

The mode of administration and dosage forms will affect the therapeutic amounts of the S. Typhi live vector or isolated recombinant outer membrane vesicles which are desirable and efficacious for the vaccination application. The current application is not limited specifically to oral administration of the vaccine, but can also include parenteral or other mucosal routes including sublingual administration as desired. The bacterial live vector materials or recombinant outer membrane vesicles are delivered in an amount capable of eliciting an immune reaction in which it is effective to increase the patient’s immune response to the expressed antigen.

The bacterial live vector vaccines or isolated recombinant outer membrane vesicles of the present invention may be usefully administered to the host animal with any other suitable pharmacologically or physiologically active agents, e.g., antigenic and/or other biologically active substances.

The attenuated S. Typhi-bacterial live vector expressing one or more antigens or isolated recombinant outer membrane vesicles described herein can be prepared and/or formulated without undue experimentation for administration to a mammal, including humans, as appropriate for the particular application. The pharmaceutical compositions may be manufactured without undue experimentation in a manner that is itself known, e.g., by means of conventional mixing, dissolving, dragee-making, levitating, emulsifying, encapsulating, entrapping, spray-drying, or lyophilizing processes, or any combination thereof.

In one embodiment, the attenuated S. Typhi-bacterial live vector expressing one or more antigens or isolated recombinant outer membrane vesicles are administered mucosally. Suitable routes of administration may include, for example, oral, lingual, sublingual, rectal, transmucosal, nasal, buccal, intrabuccal, intravaginal, or intestinal administration; intravesicular; intraurethral; administration by inhalation; intranasal, or intraocular injections, and optionally in a depot or sustained release formulation. Furthermore, one may administer the composition in a targeted drug delivery system. Combinations of administrative routes are possible.

The dose rate and suitable dosage forms for the bacterial live vector vaccine compositions or recombinant isolated outer membrane vesicles of the present invention may be readily determined by those of ordinary skill in the art without undue experimentation, by use of conventional antibody titer determination techniques and conventional bioefficacy/biocompatibility protocols. Among other things, the dose rate and suitable dosage forms depend on the particular antigen employed, the desired therapeutic effect, and the desired time span of bioactivity.

In some embodiments, the attenuated S. Typhi-bacterial live vector expressing one or more antigens or recombinant isolated outer membrane vesicles can also be prepared for nasal administration. As used herein, nasal administration includes administering the compound to the mucous membranes of the nasal passage or nasal cavity of the subject. Pharmaceutical compositions for nasal administration of the S. Typhi-bacterial live vector or recombinant isolated outer membrane vesicles include therapeutically effective amounts of the S. Typhi-bacterial live vector or recombinant isolated outer membrane vesicles prepared by well-known methods to be administered, for example, as a nasal spray, nasal drop, suspension, gel, ointment, cream or powder. Administration of the S. Typhi-bacterial live vector or isolated recombinant outer membrane vesicles may also take place using a nasal tampon or nasal sponge.

The compositions may also suitably include one or more preservatives, antioxidants, or the like. Some examples of techniques for the formulation and administration of the S. Typhi-bacterial live vector or isolated recombinant outer membrane vesicles may be found in Remington: The Science and Practice of Pharmacy, Lippincott Williams & Wilkins Publishing Co., 21^{st} addition, incorporated herein by reference.

In one embodiment, the pharmaceutical compositions contain the S. Typhi-bacterial live vector or isolated recombinant outer membrane vesicles in an effective amount to achieve their intended purpose. In one embodiment, an effective amount means an amount sufficient to prevent or treat cancer. In one embodiment, to treat means to reduce the development of, inhibit the progression of, or ameliorate the symptoms of a disease such as cancer in the subject being treated. In one embodiment, to prevent means to administer prophylactically, e.g., in the case wherein in the opinion of the attending physician the subject’s background, heredity, environment, occupational history, or the like, give rise to an expectation or increased probability that that subject is at risk of having the disease, even though at the time of diagnosis or administration that subject either does not yet have the disease or is asymptomatic of the disease.

### Therapeutic Methods

The present invention also includes methods of treating or preventing cancer in a subject, comprising administering to the subject an effective amount of a live Salmonella Typhi vector as described herein and/or an effective amount of isolated recombinant outer membrane vesicles as described herein. The present invention also includes methods of inducing an immune response in a subject. The immune response may be directed to one or more cancer antigens expressed by the Salmonella Typhi live vector.

In some embodiments, the invention provides a method of inducing an immune response in a subject in need thereof, comprising administering to the subject an immunologically-effective amount of a live Salmonella Typhi vector that has been engineered to express one or more cancer antigens, wherein the antigen is delivered to a mucosal tissue of the subject by an outer membrane vesicle produced by the Salmonella Typhi vector.

In some embodiments, the invention provides a method of inducing an immune response in a subject in need thereof, comprising administering to the subject an immunologically-effective amount of isolated recombinant outer membrane vesicles comprising one or more cancer antigens.

In some embodiments, the invention provides a method of treating or preventing cancer in a subject, comprising administering to the subject an immunologically-effective amount of isolated recombinant outer membrane vesicles comprising one or more cancer antigens.

The route of administering the recombinant outer membrane vesicles is not limiting. In some embodiments, the recombinant outer membrane vesicles are administered intranasally, mucosally, orally, parenterally, intravenously, intramuscularly, intradermally, topically, or a combination thereof.

In one embodiment, the recombinant outer membrane vesicles used in the methods herein comprise one or more cancer antigens comprising CEA or a fragment or variant thereof, MUC1 or a fragment or variant thereof, or a fusion protein comprising CEA or a fragment or variant thereof and MUC1 or a fragment or variant thereof. In some embodiments, the antigen is fused to a surface presentation protein such as Lpp-OmpA.

In some embodiments, the invention provides a method of inducing an immune response in a subject in need thereof, comprising administering to the subject an immunologically-effective amount of isolated recombinant outer membrane vesicles from Salmonella Typhi comprising one or more cancer antigens, wherein the Salmonella Typhi has been engineered to express the antigen, wherein the outer membrane vesicle is delivered to a mucosal tissue of the subject.

In one embodiment, the method comprises administering a combination of live Salmonella Typhi vectors of the invention to a subject. In some embodiments, the combination of vectors is present in the same composition. In some embodiments, the vectors are present in separate compositions.

In one embodiment, the method comprises administering a combination of isolated recombinant outer membrane vesicles to a subject.

In some embodiments, the live Salmonella Typhi vectors or isolated recombinant outer membrane vesicles of the invention are administered to a subject with cancer. In some embodiments, the live Salmonella Typhi vectors or isolated recombinant outer membrane vesicles of the invention are administered to a subject at risk of developing cancer. In some embodiments, the live Salmonella Typhi vectors or isolated recombinant outer membrane vesicles are administered to the subject with one or more additional therapies to treat the cancer. In some embodiments, the one or more additional therapies are selected from chemotherapy, radiation, surgery, and immunotherapy. In a preferred embodiment, the live Salmonella Typhi vectors or isolated recombinant outer membrane vesicles are co-administered to the subject with one or more additional therapies as soon as possible after early detection of the target cancer. It is now well appreciated that early detection will enhance the odds of successful treatment of solid tumors prior to progression to large solid masses that can dramatically reduce access of target cancer antigens to therapeutic treatment of any kind.

In some embodiments, the Salmonella Typhi vectors and/or recombinant outer membrane vesicles are administered to a subject following early detection of cancer. This is advantageous because early detection and intervention can improve the probability of curing the cancer in the subject. In some embodiments, the method comprises a diagnostics screening method to detect the presence of cancer in the subject. Such diagnostic screening methods can include imaging and/or screening tissue or blood samples from the subject for the presence of cancer cells or markers of the cancer. Suitable early diagnostic methodologies are described, for example, in Cohen et al. Science, 359: 926-930 (2018) and Lennon et al., Science 10.1126/science.abb9601 (2020), which are incorporated by reference herein in their entirety.

In some embodiments, cancer can be detected by conducting a liquid biopsy, for example, by taking a blood sample and detecting cancer cells or makers in the sample. In some embodiments, a positive blood test detecting a cancer can be confirmed by scanning, e.g., PET-CT scanning, to identify a tumor mass. In some embodiments, protein or nucleic acid markers are detected in the liquid sample to identify the presence of a cancer in the subject. The type of cancer that can be identified in blood is not necessarily limiting. Such cancers can include lung cancer, ovarian cancer, colorectal cancer, breast cancer, lymphoma, kidney cancer, thyroid cancer, uterine cancer, and cancer of the appendix.

In some embodiments, cancer of the ovary can be detected in blood by detecting the presence of marker TP53, CA19-9, CA125, CA15-3, or a combination thereof.

In some embodiments, cancer of the lung can be detected in blood by detecting the presence of marker KRAS, TP53, CA15-3, HGF, CEA, EGFR, PIK3CA, or a combination thereof.

In some embodiments, cancer of the uterus can be detected in blood by detecting the presence of marker TP53, CA19-9 or a combination thereof.

In some embodiments, cancer of the thyroid can be detected in blood by detecting the presence of marker CEA.

In some embodiments, colorectal cancer can be detected in blood by detecting the presence of marker BRAF, TP53 or a combination thereof.

In some embodiments, breast cancer can be detected in blood by detecting the presence of marker PIK3CA, TP53 or a combination thereof.

In some embodiments, lymphoma can be detected in blood by detecting the presence of marker HGF, NRAS or a combination thereof.

In some embodiments, kidney can be detected in blood by detecting the presence of marker KRAS.

In some embodiments, cancer of the appendix can be detected in blood by detecting the presence of marker CEA.

Vaccine strategies are well known in the art and therefore the vaccination strategy encompassed by the invention does not limit the invention in any manner. In certain aspects of the invention, the S. Typhi live vector vaccine expressing one or more cancer antigens or isolated recombinant outer membrane vesicles is administered alone in a single application or administered in sequential applications, spaced out over time.

In other aspects of the invention, the S. Typhi live vector vaccine is administered as a component of a heterologous prime/boost regimen. “Heterologous prime/boost” strategies are 2-phase immunization regimes involving sequential administration (in a priming phase and a boosting phase) of the same antigen in two different vaccine formulations by the same or different route. In particular aspects of the invention drawn to heterologous prime/boost regimens, a mucosal prime/parenteral boost immunization strategy is used. For example, one or more S. Typhi live vector vaccines as taught herein can be administered orally or via another mucosal route and subsequently boosted parentally with a vaccine composition comprising isolated recombinant outer membrane vesicles from a S. Typhi vector comprising one or more of the cancer antigens.

In another aspect, the present invention is directed to methods of inducing an immune response against an antigen in a subject in need thereof, comprising administering to the subject an immunologically-effective amount of a live Salmonella Typhi vector of the invention as a prime, and subsequently administering a boost composition comprising a composition comprising isolated recombinant outer membrane vesicles from a S. Typhi vector comprising one or more of the cancer antigens.

In some embodiments, the isolated recombinant outer membrane vesicles of the invention are administered as a prime and is boosted with the S. Typhi live vector vaccine of the invention. In some embodiments, the boost is administered mucosally, e.g., orally, or parenterally.

In some embodiments, in the context of heterologous prime/boost regimens, the subject is administered:
i. a live Salmonella Typhi vector that has been engineered to express one or more cancer antigens; and a lipid A deacylase PagL or a fragment or variant thereof; and
ii. isolated recombinant outer membrane vesicles that have been isolated from a live Salmonella Typhi vector that has been engineered to express the one or more cancer antigens; a lipid A deacylase PagL or a fragment or variant thereof; and an outer membrane folding protein BamA or a fragment or variant thereof. In some embodiments, the live Salmonella Typhi vector of part i. is administered as a prime and the isolated recombinant outer membrane vesicles of part ii. is administered as a boost. In some embodiments, the isolated recombinant outer membrane vesicles of part ii. is administered as a prime and the live Salmonella Typhi vector of part ii. is administered as a boost.

As used herein, an “immune response” is the physiological response of the subject’s immune system to an immunizing composition. An immune response may include an innate immune response, an adaptive immune response, or both. In one embodiment of the present invention, the immune response is a protective immune response. A protective immune response confers immunological cellular memory upon the subject, with the effect that a secondary exposure to the same or a similar antigen is characterized by one or more of the following characteristics: shorter lag phase than the lag phase resulting from exposure to the selected antigen in the absence of prior exposure to the immunizing composition; production of antibody which continues for a longer period than production of antibody resulting from exposure to the selected antigen in the absence of prior exposure to the immunizing composition; a change in the type and quality of antibody produced in comparison to the type and quality of antibody produced upon exposure to the selected antigen in the absence of prior exposure to the immunizing composition; a shift in class response, with IgG antibodies appearing in higher concentrations and with greater persistence than IgM, than occurs in response to exposure to the selected antigen in the absence of prior exposure to the immunizing composition; an increased average affinity (binding constant) of the antibodies for the antigen in comparison with the average affinity of antibodies for the antigen resulting from exposure to the selected antigen in the absence of prior exposure to the immunizing composition; and/or other characteristics known in the art to characterize a secondary immune response.

In a further embodiment, the method of inducing an immune response comprises administering a pharmaceutical formulation as provided herein comprising one or more Salmonella Typhi live vectors or isolated recombinant outer membrane vesicles of the present invention to a subject in an amount sufficient to induce an immune response in the subject (an immunologically-effective amount). In some embodiments, the compositions are administered intranasally.

In some embodiments, one or more S. Typhi live vector vaccines or isolated recombinant outer membrane vesicles of the invention are mucosally administered in a first priming administration, followed, optionally, by a second (or third, fourth, fifth, etc.... ) priming administration of the live vector vaccine or isolated recombinant outer membrane vesicles from about 2 to about 10 weeks later. In some embodiments, a boosting composition is administered from about 3 to about 12 weeks after the priming administration. In some embodiments, the boosting composition is administered from about 3 to about 6 weeks after the priming administration. In some embodiments, the boosting composition is substantially the same type of composition administered as the priming composition (e.g., a homologous prime/boost regimen).

In practicing immunization protocols for treatment and/or prevention, an immunologically-effective amount of a live Salmonella Typhi vector or isolated recombinant outer membrane vesicles is administered to a subject. As used herein, the term “immunologically-effective amount” means the total amount of a live S. Typhi vector or isolated recombinant outer membrane vesicles that is sufficient to show an enhanced immune response in the subject. When “immunologically-effective amount” is applied to an individual therapeutic agent administered alone, the term refers to that therapeutic agent alone. When applied to a combination, the term refers to combined amounts of the ingredients that result in the therapeutic effect, whether administered in combination, serially or simultaneously.

The particular dosage depends upon the age, weight, sex and medical condition of the subject to be treated, as well as on the method of administration. Suitable doses can be readily determined by those of skill in the art.

The dose of the recombinant outer membrane vesicles that is administered is not necessarily limiting. The dose of recombinant outer membrane vesicles can be administered one or more times, spaced out over time.

In some embodiments, the dose is adjusted up or down over time. In some embodiments, a dose of from about 0.001 µg/kg to about 10 µg/kg of recombinant outer membrane vesicles is administered to the subject, based on the subject’s body weight (in kg). In some embodiments, a dose of from about 0.1 µg/kg to about 10 µg/kg of recombinant outer membrane vesicles is administered to the subject. In some embodiments, a dose of from about 0.25 µg/kg to about 5.0 µg/kg of recombinant outer membrane vesicles is administered to the subject. In some embodiments, a dose of from about 0.25 µg/kg to about 2.5 µg/kg of recombinant outer membrane vesicles is administered to the subject. In some embodiments, a dose of from about 0.25 µg/kg to about 1.0 µg/kg of recombinant outer membrane vesicles is administered to the subject.

In some embodiments, the recombinant outer membrane vesicles are administered via a parenteral route. In some embodiments, the route of administration is intravenous. In some embodiments, the route of administration is intramuscular.

In some embodiments, the subject is administered multiple doses of the recombinant outer membrane vesicles (or multiple doses of live Salmonella Typhi vector, or a combination of recombinant outer membrane vesicles and live Salmonella Typhi vector) separated by a period of time. In some embodiments, the subject is administered two doses, three doses, four doses, five doses, six doses or more. In some embodiments, the doses are spaced from about 2 days-12 weeks apart. In some embodiments, the doses are spaced from about 2-7 days apart. In some embodiments, the subject is administered from 2-5 doses spaced 2-7 days apart. In some embodiments, the doses are spaced from about 1-6 weeks apart. In some embodiments, the doses are spaced about one week apart. In some embodiments, the doses are spaced about two weeks apart. In some embodiments, the doses are spaced about three weeks apart.

In some embodiments, the subject is administered a priming dose of recombinant outer membrane vesicles administered parenterally, e.g., intravenously or intramuscularly, followed by a mucosally administered, e.g., intranasal, boosting dose of the live Salmonella Typhi vector, e.g., from about 1-4 weeks later.

In some embodiments, the subject is administered a priming dose of live Salmonella Typhi vector administered mucosally, e.g., intranasal, followed by a parenterally, e.g., intravenously or intramuscularly, administered boosting dose of the recombinant outer membrane vesicles, e.g., from about 1-4 weeks later.

In some embodiments, the subject is administered a priming dose of recombinant outer membrane vesicles administered parenterally, e.g., intravenously or intramuscularly, followed by a parenterally, e.g., intravenously or intramuscularly, administered boosting dose of the recombinant outer membrane vesicles, e.g., from about 1-4 weeks later.

In some embodiments, the subject is administered a priming dose of live Salmonella Typhi vector administered mucosally, e.g., intranasal, followed by a mucosally administered, e.g., intranasal, boosting dose of the live Salmonella Typhi vector, e.g., from about 1-4 weeks later.

In some embodiments, the S. Typhi-bacterial live vector can be administered intranasally at a dose of about 1 × 10⁹ CFD. In some embodiments, the attenuated S. Typhi-bacterial live vector can be administered intranasally at a dose of about 1 × 10⁸ CFU to about 1 × 10¹⁰ CFU.

The term “subject” as used herein, refers to animals, such as mammals. For example, mammals contemplated include humans, primates, dogs, cats, sheep, cattle, goats, pigs, horses, mice, rats, rabbits, guinea pigs, and the like. The terms “subject,” “patient,” and “host” are used interchangeably.

In some embodiments, the subject is administered a multivalent recombinant outer membrane vesicle following either early diagnosis of colorectal cancer, or a subject in the early stages of solid tumor progression with sufficient blood vessel density to potentially allow for passive deposition of recombinant outer membrane vesicles. Such a patient population could be identified through the use of newly developed blood-based diagnostic assays (Lennon et al., Science, (2020), 369(6499). In some embodiments, subjects with more advanced stage III cancers, recovering from post-operative surgery and completion of adjuvant systemic chemotherapy, are treated with the recombinant outer membrane vesicles, either alone in multiple administrations or in combination with a VLP-CEA vaccine (or the live Salmonella vector herein) administered in a heterologous prime-boost regime. In some embodiments, a subject that is administered recombinant outer membrane vesicles has an increased risk for colorectal or other cancers in which MUC1 is often overexpressed (see, e.g., Crosby et al., Journal for immunotherapy of cancer, (2020), 8(2); Kimura et al., Cancer Prev Res (Phila) 2013; 6(1): 18-26; Lohmueller et al., Sci Rep 2016; 6: 31740).

The live Salmonella Typhi vectors or isolated recombinant outer membrane vesicles of the invention may be administered to warm-blooded mammals of any age. The live Salmonella Typhi vectors can be administered as a single dose or multiple priming doses, followed by one or more boosters. For example, a subject can receive a single dose, then be administered a booster dose up to 1 month, 2 months, 3 months, 4 months, 5 months, 6 months, 9 months, 1 year, 2 years, 3 years, 4 years, 5 years, 6 years, 7 years, 8 years, 9 years, or 10 or more years later.

Application of the teachings of the present invention to a specific problem is within the capabilities of one having ordinary skill in the art in light of the teaching contained herein. Examples of the compositions and methods of the invention appear in the following non-limiting Examples.

### EXAMPLES

### Example 1 Development of a PagL-Mediated Antigen Delivery Platform

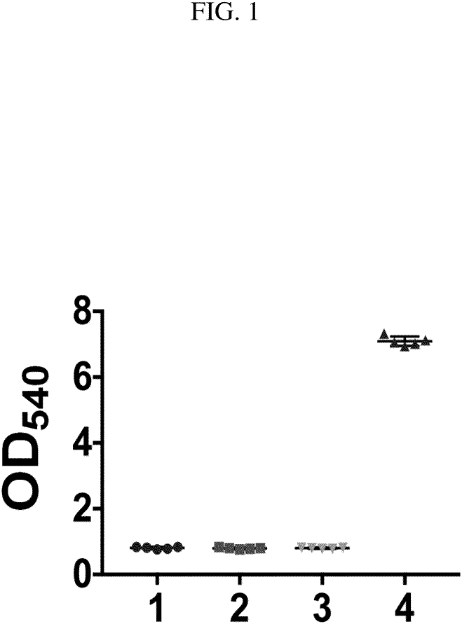

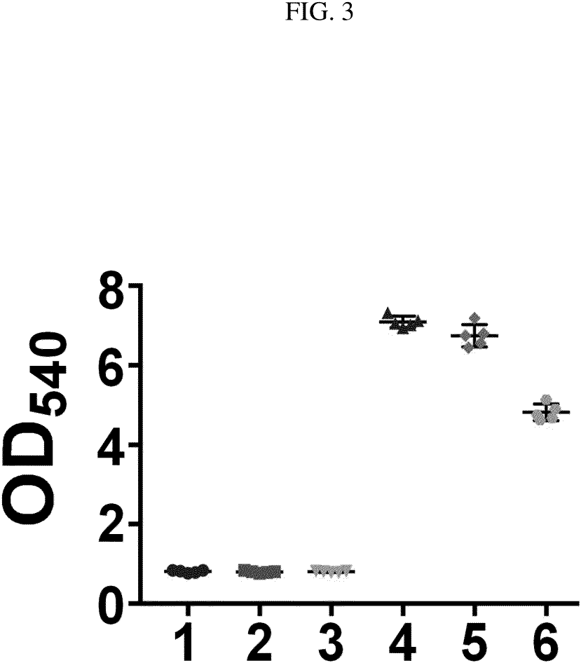

Because ClyA is a hemolysin with cytopathic characteristics that may reduce the clinical acceptability of candidate vaccine strains in which ClyA is over-expressed, we sought to develop a non-pathogenic alternative for inducing formation and export of OMVs based on PagL (Ludwig et al., Mol Microbiol 1999; 31(2): 557-67.; Lai et al., Infect Immun 2000; 68(7): 4363-7). We therefore constructed a synthetic pagL gene and inserted it into our non-antibiotic low-copy-number expression plasmid pSEC10, replacing the clyA gene to create pPagL. As with our previous experiments with inducible outer membrane vesicles, we wished to monitor OMV export by measuring the hemolytic activity associated with ClyA-mediated vesiculation. Therefore, we integrated a cassette encoding ClyA into the guaBA locus of CVD 910 and then introduced pPagL into the resulting strain to create CVD 910ΔguaBA::clyA(pPagL). Note that in this particular strain, ClyA is acting as a surrogate hemolytic reporter for a chromosomally encoded antigen, with over-expression of plasmid-encoded PagL expected to significantly improve rOMV export. All strains were grown at 37° C. into early-log phase growth, and hemolytic activity was measured at OD₅₄₀ for approximately 2 × 10⁷ CFU of bacteria against sheep red blood cells. As shown in FIG. 1 no hemolytic activity was present in the vaccine strain CVD 910 as expected (lane 2). Surprisingly, the hemolytic activity of chromosomally encoded ClyA was not detected in CVD 910ΔguaBA::clyA (lane 3), due to the drop in copy number versus plasmid-encoded hemolytic activity observed for CVD 910(pSEC10) [see FIG. 3, lane 3]. However, significant hemolytic activity was observed when pPagL was introduced into 910ΔguaBA::clyA (lane 4), clearly demonstrating that over-expression of PagL induces excellent export of outer membrane proteins (i.e. ClyA in this case) via outer membrane vesicles.

Summary of Studies. Taken together, our results firmly establish the feasibility of developing an attenuated S. Typhi-based mucosal live vector vaccine that can efficiently express and deliver properly folded foreign proteins to the surface of our live vector vaccine. These foreign antigens can be expressed from chromosomally integrated gene cassettes which will allow construction of a live vector vaccine that does not require large and potentially unstable multicopy expression plasmids for delivery of antigens. We have also engineered a unique outer membrane vesicle antigen delivery platform and successfully completed proof-of-principle studies demonstrating the efficiency of a PagL-mediated antigen delivery system using ClyA as a model outer membrane protein for export via recombinant rOMVs.

### Example 2 Development of a PagL-Mediated Antigen Delivery Platform

We constructed three synthetic pagL gene alleles, designated pagL v1 (SEQ ID NOS: 1 and 2), pagL v2 (SEQ ID NOS: 3 and 4), and pagL v3 (SEQ ID NOS: 5). These 3 versions differ in the 5′-terminal DNA sequences controlling the translation efficiency of each allele; this cautious engineering approach was adopted because the optimal translation efficiency of pagL assuring sufficient synthesis of biologically active PagL, while avoiding potentially lethal over-expression of this protein, was unknown at the time of these experiments. The amino acid sequence of pagL v2 and v3 is identical. To this end, pagL v1 carries an optimized ribosome binding site (RBS), an ATG start codon, and several optimized codons codon at the beginning of the gene to enhance translation efficiency. pagL v2 is similar to v1 but contains a GTG start codon to slightly reduce translation efficiency. pagL v3 is essentially identical to the wild type chromosomal sequence of the pagL gene naturally present within Salmonella enterica serovar Typhimurium. Therefore, we expected the highest levels of PagL synthesis from v1, with decreasing levels of synthesis from v2 and the lowest levels of synthesis from v3.

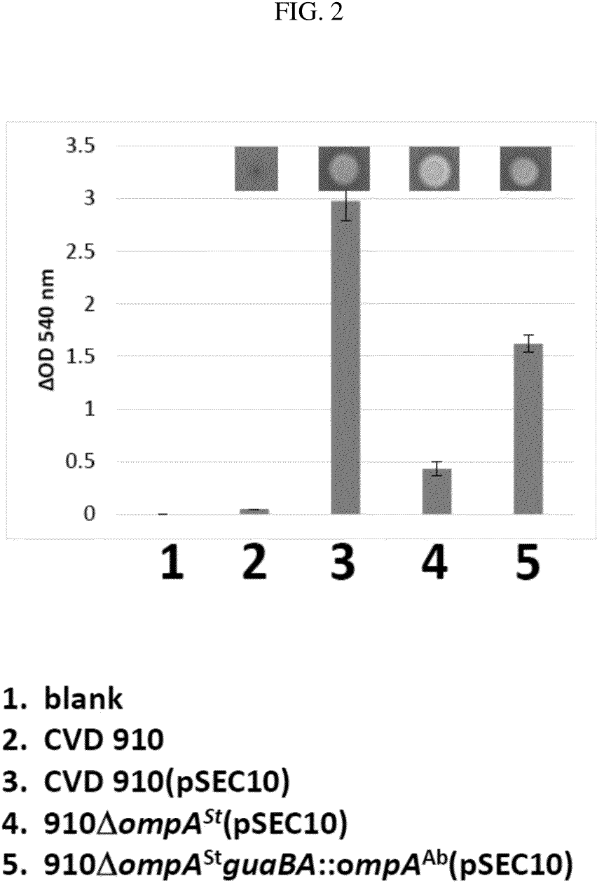

Each cassette was inserted as a BamHI-NheI fragment into our non-antibiotic low-copy-number expression plasmid pSEC10 digested with BamHI and NheI, replacing the clyA gene to create pPagL; the expected sequence of pPagL v1 is listed in SEQ ID NO:6. As with our previous experiments with inducible recombinant outer membrane vesicles (rOMVs), we wished to monitor OMV export by measuring the hemolytic activity associated with ClyA-containing vesicles. Therefore, we integrated a cassette encoding ClyA into the guaBA locus of CVD 910 and then introduced pPagL into the resulting strain to create CVD 910DguaBA::clyA(pPagL). Note that in this particular strain, ClyA is acting as a surrogate hemolytic reporter for a chromosomally encoded antigen protein, with over-expression of plasmid-encoded PagL expected to significantly improve rOMV export. All strains were grown at 37° C. into early-log phase growth, and hemolytic activity was measured at OD₅₄₀ for approximately 2 × 10⁷ CFU of bacteria against sheep red blood cells. As shown in FIG. 2, no hemolytic activity was present in the vaccine strain CVD 910 as expected (lane 2). Surprisingly, the hemolytic activity of chromosomally encoded ClyA was not detected in CVD 910DguaBA::clyA (lane 3), due to the drop in copy number versus plasmid-encoded hemolytic activity observed for CVD 910(pSEC10). However, striking hemolytic activity was observed when pPagL was introduced into 910DguaBA::clyA (lane 4), clearly demonstrating that over-expression of PagL induces excellent export of rOMVs (containing ClyA as the surrogate outer membrane protein in this case).

### Example 3 Development of an Inducible Vesicle Delivery System

We have genetically engineered a novel osmotically inducible vaccine antigen delivery system in which foreign antigens, expressed on the outer membrane surface of our attenuated Salmonella enterica serovar Typhi candidate vaccine strain CVD 910, can be efficiently exported off the surface of the vaccine strain via recombinant outer membrane vesicles carrying these foreign surface-expressed protein antigens. To test this concept in the context of vaccine development, we further engineered our first prototype live attenuated CVD 910 candidate vaccine in which an outer membrane protein is efficiently expressed on the surface of the live strain. We have introduced low copy expression plasmids encoding inducible over-expression of PagL, a novel outer membrane lipid A deacylase recently reported to catalyze hypervesiculation when over-expressed in Salmonella; we hypothesized that over-expression of PagL could catalyze the formation of rOMVs carrying antigen, for efficiently delivery to immune inductive sites to elicit protection against disease (Elhenawy et al., mBio 2016; 7(4): pii: e00940-16. doi: 10.1128/mBio.-16.). To improve the efficiency of transport of both antigen and PagL to the outer membrane (with the intent of enhancing rOMV-mediated antigen export and improving vaccine efficacy), we also integrated into the chromosome of this prototype vaccine an inducible genetic cassette encoding the outer membrane folding protein BamA.

BamA is an ~90 kDa protein that constitutes an essential component of a 5-protein outer membrane β-barrel assembly machinery (BAM) complex that catalyzes the insertion of β-barrel proteins into the outer membrane of Gram negative bacteria (Noinaj et al., Nature reviews Microbiology 2017; 15(4): 197-204.). However, the complete BamABCDE complex is not required for the efficient insertion of select outer membrane proteins; indeed, it has been reported by several groups that OmpA can be efficiently incorporated into lipid bilayers in which only BamA is present (Gessmann et al., Proc Natl Acad Sci U S A 2014; 111(16): 5878-83.; Plummer et al., Biochemistry 2015; 54(39): 6009-11.). We therefore hypothesized that over-expression of BamA in a vaccine strain coexpressing PagL as well would lead to more efficient export of outer membrane vesicles carrying surface-expressed foreign antigens. Considering a recent report in which purified AbBamA from A. baumannii conferred protection in mice against challenged with MDR A. baumannii, we chose to use a bamA^{Ab} allele from A. baumannii (Singh et al., Sci Rep 2017; 7(1): 12411).

Development of a PagL-mediated antigen delivery platform. Having demonstrated expression of antigen on the surface of our candidate vaccine strain CVD 910, we then began development of an inducible outer membrane vesicle antigen export system for delivery of surface expressed antigen to immune inductive sites after immunization. To accomplish this, we focused on the use of PagL, a lipid A deacylase recently reported to catalyze hypervesiculation when over-expressed in Salmonella (Elhenawy et al., mBio 2016; 7(4): pii: e00940-16. doi: 10.1128/mBio.-16.). Given that over-expression of PagL could theoretically induce hypervesiculation of antigen-containing rOMVs, this strategy also presented the unique opportunity of using purified multivalent rOMVs either as mucosal vaccines by themselves or in combination with live carrier vaccines from which they were purified.

To investigate this intriguing possibility, we first attempted to monitor OMV export by phenotypically tagging vesicles with a novel endogenous Salmonella hemolysin called cytolysin A (ClyA), first reported by Wai et al. to catalyze the formation of large outer membrane vesicles when over-expressed (Wai et al., Cell 2003; 115(1): 25-35.). Use of this simple hemolytic reporter phenotype allowed quick quantitative evaluation of OMV export to efficiently guide optimization of expression cassettes and avoid potentially lethal over-expression of vesiculating proteins; we have successfully exploited expression of ClyA for export of foreign antigens out of engineered carrier strains as fusion proteins encoded by low copy expression plasmids (Galen et al., Infect Immun 2004; 72(12): 7096-106.). However, for testing PagL-mediated vesiculation, we needed to lower expression of the ClyA hemolysin such that ClyA was exported to the surface at levels sufficient to detect hemolytic activity (i.e to phenotypically “tag” the membrane surface), but not high enough to actually catalyze ClyA-mediated vesicle formation. Therefore, we integrated a cassette encoding ClyA into the guaBA locus of CVD 910 creating the reporter strain CVD 910ΔguaBA::clyA. We then constructed 3 versions of a synthetic pagL gene in which the translation efficiency varied with the distance of a consensus ribosome binding site (AGGAGG) 5 bases upstream from an optimum ATG start codon (pagLv1), 6 bases upstream of a less efficient GTG start codon (pagLv2), or 5 bases upstream of this GTG start codon pagLv3); given that the ideal positioning of an RBS is 7-9 bases away from an ATG start codon, we expected decreasing expression levels of these 3 isogenic alleles in the order pagLv1>pagLv2>pagLv3 (Ringquist et al., Mol Microbiol 1992; 6(9): 1219-29.). Each allele was inserted into a low-copy-number expression plasmid pSEC10, downstream of an osmotically controlled P_{ompC} promoter to create pPagLv1, pPagLv2, and pPagLv3 respectively; inducible expression of PagL in the resulting expression plasmids is transcriptionally controlled by osmotic induction of the ompC promoter (Stokes et al., Infect Immun 2007; 75(4): 1827-34.; Galen et al., Infect Immun 2010; 78(1): 337-47.; Galen et al., Infect Immun 1999; 67(12): 6424-33.) We hypothesized that as expression of plasmid-encoded PagL increased with the efficiency of the RBS, export of ClyA-tagged rOMVs would also increase, accompanied by an increase in hemolytic activity.

To test this hypothesis, each plasmid was introduced into the reporter strain CVD 910ΔguaBA::clyA. Strains were then grown under inducing conditions at 37° C. into early-log phase growth, and hemolytic activity was measured at OD₅₄₀ for approximately 2 x 10⁷ CFU of bacteria against sheep red blood cells. As shown in FIG. 3, no hemolytic activity was present in the vaccine strain CVD 910 (lane 2). As expected, the hemolytic activity of chromosomally encoded ClyA was not detected in CVD 910ΔguaBA::clyA (lane 3), due to reduced expression levels from the chromosome. However, significant hemolytic activity was observed for 910ΔguaBA::clyA(pPagLv1), which decreased with the engineered efficiency of the RBS (lane 4 versus lanes 5 and 6), supporting the hypothesis that over-expression of PagL induces excellent export of outer membrane proteins (i.e. ClyA in this case) via outer membrane vesicles.

Enhancing surface expression of OMPs by over-expression of AbBamA. The preliminary results suggested that although antigen was successfully expressed on the surface of CVD 910, expression from non-permeabilized cells was decreased versus levels detected in permeabilized cells. We hypothesized that this disparity might be due to the rate of transport and/or proper insertion of proteins into the outer membrane, and that over-expression of a transport protein affecting translocation rates might enhance surface expression. We noted that the antigen and PagL are both β-barrel transmembrane proteins (Rutten et al., Proc Natl Acad Sci U S A 2006; 103(18): 7071-6.). Insertion of βbarrel proteins into the outer membrane of Gram-negative bacteria is mediated by the βbarrel assembly (BAM) complex, of which the protein BamA (itself a β-barrel protein) comprises the essential core component (Albrecht et al., Acta Crystallogr D Biol Crystallogr 2014; 70(Pt 6): 1779-89.; Noinaj et al., Nature reviews Microbiology 2017; 15(4): 197-204.). It has also been reported that BamA alone can accelerate outer membrane folding and membrane insertion in vitro of β-barrel proteins. We therefore hypothesized that over-expression of BamA may be able to improve surface expression of outer membrane proteins including an exemplary vaccine antigen; conceivably, enhanced transport of PagL to the outer membrane could also enhance rOMV formation and hence foreign antigen delivery to immune inductive sites.

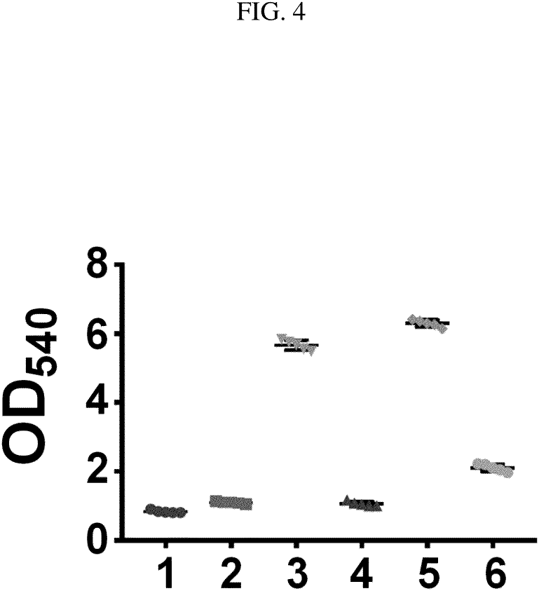

To test this hypothesis, we engineered synthetic gene cassettes encoding AbBamA. Interestingly, our original cassettes in which translation was initiated with an ATG start codon were never successfully inserted into low copy expression plasmids. To avoid potentially lethal over-expression of AbBamA, we therefore engineered ribosome binding sites positioned 5 bases (bamA^{Ab}v1) or 4 bases bamA^{Ab}v2) upstream of a GTG start codon to more tightly control translation levels; given that the ideal positioning of an RBS is 7-9 bases away from the start codon, we expected bamA^{Ab}v1 to have slightly higher expression levels than bamA^{Ab}v2 (Ringquist et al., Mol Microbiol 1992; 6(9): 1219-29.). As with the pagL alleles, we engineered the bamA alleles under the transcriptional control of a P_{ompC} promoter and inserted the resulting cassettes into our low copy expression plasmid to create pAbBamAv1 and pAbBamAv2. These plasmids were then introduced into CVD 910ΔguaBA::clyA. Although ClyA does not possess a β-barrel structure, we wanted to investigate any potential effect of AbBamA over-expression on OMV formation (Wallace et al., Cell 2000; 100: 265-76.). As summarized in FIG. 4, the hemolytic activity for plasmid-based expression of ClyA in CVD 910(pSEC10) was markedly higher than that observed for chromosomally encoded ClyA in CVD 910ΔguaBA::clyA due to enhanced copy number of clyA in CVD 910(pSEC10) (lane 3 versus lane 4). Surprisingly, introduction of pAbBamAv1 into CVD 910ΔguaBA::clyA was able to enhance hemolytic activity to levels comparable to plasmid-based expression in CVD 910(pSEC10), an effect that was reduced in strains carrying the less efficiently expressing bamA^{Ab}v2 allele (lane 5 versus lane 6). We conclude from these experiments that AbBamA can enhance the formation of outer membrane vesicles, phenotypically tagged with ClyA and exported from CVD 910, and may also enhance the export of vesicles carrying the antigen or other foreign antigens relevant to vaccine development.

Encouraged by successfully demonstrating surface expression of antigen in our candidate vaccine strain CVD 910, as well as also demonstrating the capacity of both PagL and AbBamA to enhance export of rOMVs, we then tested the hypothesis that antigen surface expression could be optimized by co-expression of both PagL and AbBamA in a single vaccine strain; given that surface expressed outer membrane proteins are not instantly exported via vesicles, we reasoned that as surface expression increased, outer membrane vesicle formation would also eventually increase, although not explicitly determined in these preliminary experiments. To accomplish this, we integrated the osmotically controlled P_{ompC}-bamA^{Ab}v1(SEQ ID NO:7), encoding the 93.2 kDa AbBamA protein (SEQ ID NO:8), into the guaBA locus of CVD 910; we then introduced the pAntigen expression plasmid into the resulting strain, creating CVD 910ΔguaBA::bamA^{Ab}v1(Antigen).

These results firmly establish the feasibility of developing an attenuated S. Typhi-based mucosal live carrier vaccine that can efficiently express and deliver properly folded foreign outer membrane proteins to the surface of our carrier vaccine. To improve the clinical acceptability of our candidate live carrier vaccine, we have formally excluded any effect of antigen expression on the virulence of our live carrier. We have also engineered a unique PagL-mediated outer membrane vesicle antigen delivery platform in which the efficiency of antigen surface expression is enhanced by over-expression of the outer membrane folding protein AbBamA; this innovative modification improves surface expression of outer membrane proteins. We conclude that enhanced surface expression catalyzed by AbBamA will also enhance surface expression of other surface-targeted foreign proteins; induction of PagL will then catalyze the efficient export of recombinant OMVs potentially carrying a wide variety of foreign proteins from either prokaryotic or eukaryotic organisms. This technology is not limited to vaccine development against human pathogens but can also be used in veterinary and other applications, such as the development of immunotherapeutic vaccines against solid tumors as well (Niethammer et al., BMC Cancer 2012; 12: 361; Schmitz-Winnenthal et al., Oncoimmunology 2015; 4(4): e1001217.; Schmitz-Winnenthal et al., Oncoimmunology 2018; 7(4): e1303584).

### Example 4. Development of a Candidate Bivalent S. Typhi-Based Colorectal Cancer Vaccine

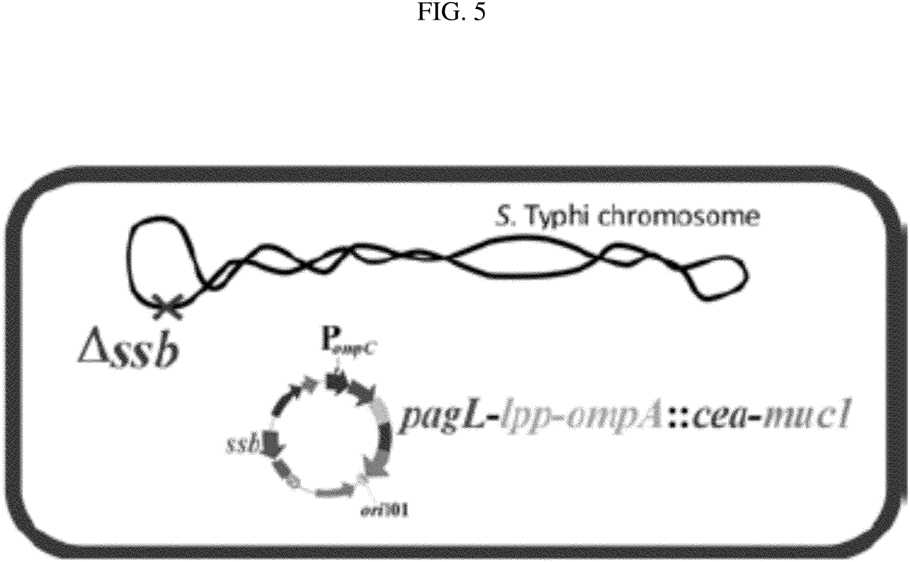

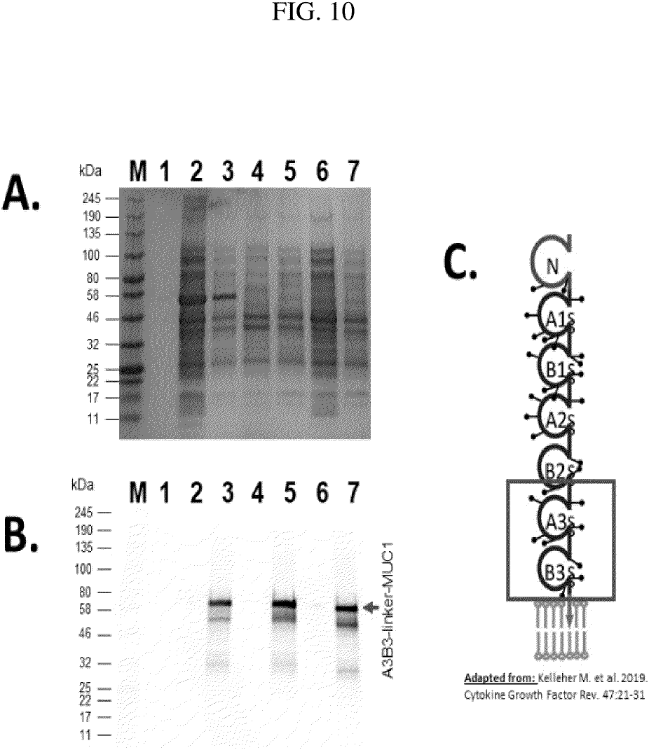

Using previously attenuated and highly immunogenic S. Typhi-based carrier vaccine strains, we have recently engineered and functionally tested an osmotically inducible and highly efficient recombinant outer membrane vesicle (rOMV) antigen delivery system driven by over-expression of the lipid A deacylase PagL (Galen et al., J Infect Dis 2009; 199(3): 326-35; Galen et al., Vaccine 2014;32(35): 4376-85; Galen et al., Infect Immun 2015; 83(1): 161-72; Elhenawy et al., mBio 2016; 7(4): pii: e00940-16. doi:10.1128/mBio.-16). PagL is encoded by a genetically stabilized low copy number expression plasmid, stabilized through trans-complementation of an otherwise lethal chromosomal deletion of the single stranded binding protein (SSB) (FIG. 5) (Galen et al., Infect Immun 2010; 78(1): 337-47.). We have exported (see FIG. 10) a bivalent fusion protein, encoding domains from CEA and MUC-1, to the surface of our carrier vaccine using the chimeric Lpp-OmpA surface display peptide (Francisco et al., Proc Natl Acad Sci U S A 1992; 89(7): 2713-7.; Hui et al., Biotechnol Lett 2019; 41(6-7): 763-77.). Subsequent osmotic induction in vivo of our orally administered recombinant carrier vaccine will result in presentation of our targeted colorectal-associated antigens via highly immunogenic and efficiently expressed rOMVs bearing a surface expressed CEA-MUC-1 fusion protein.

### Example 5 Cancer Vaccine Cassettes

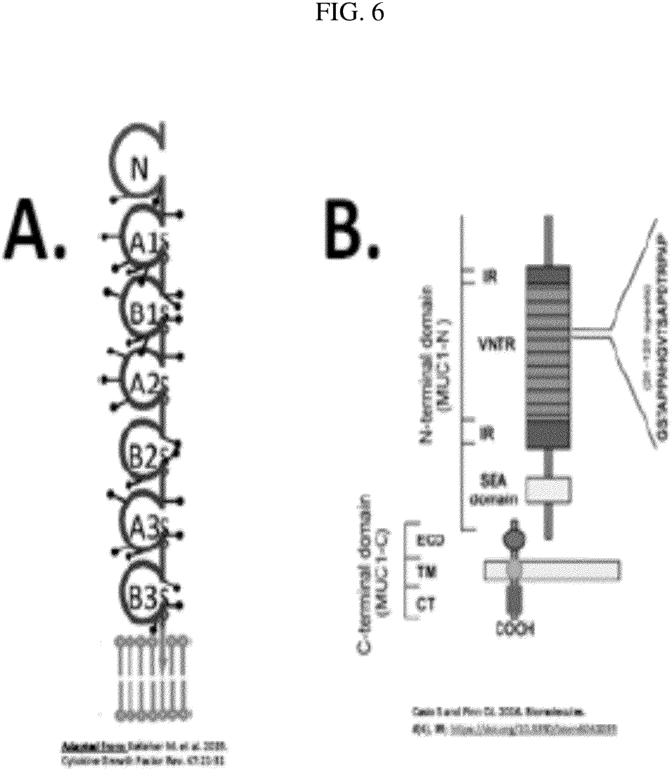

Here we describe the design of two master synthetic gene cassettes encoding fusion proteins intended to function as antigens for vaccination against colorectal cancer (FIG. 6). These synthetic cassettes are intended to be synthesized such that the cassette is flanked by the restriction enzyme BamHI on the 5′-terminus and by SpeI at the 3′-terminus of the cassette; as such, this cassette is intended in a preferred embodiment to be inserted downstream of the PagL open reading frame of the patented expression plasmid pPagL, such that the resulting plasmid encodes an operon comprised of pagL and this fusion gene, transcriptionally controlled by the osmotically regulated P_{ompC} promoter. Upon osmotic induction of this vaccine plasmid, the targeted cancer antigens will be expressed on the surface of the S. Typhi-based carrier vaccine, followed by export of these antigens via outer membrane vesicles induced by co-expression of PagL.

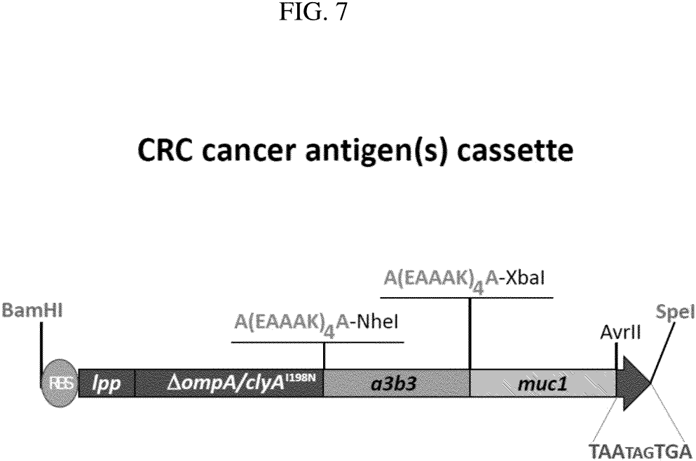

The two intended fusion proteins to be co-expressed with PagL are each comprised of a surface expression cassette operationally linked to two additional cancer antigen cassettes, each separated by an engineered linker region [designated as A(EAAAK)₄A] (FIG. 7); the linker region is designed to fold into a rigid alpha helix which will separate each cancer domain to allow proper folding after translation (Chen et al., Advanced drug delivery reviews 2013; 65(10): 1357-69). The surface expression cassette is composed of either a modified and patented non-hemolytic version of the ClyA protein (designated here as clyA^{I198N}) or a previously published surface expression cassette designated lpp-ompA (designated here as LOA) (Francisco el al., Proc Natl Acad Sci U S A 1992; 89(7): 2713-7). These surface expression cassettes are in turn operationally linked to a cassette encoding a cancer fusion protein comprised of two domains, one from the cancer antigen carcinoembryonic antigen (CEA) and the other from MUC1 (FIG. 7). The domain from CEA is designated A3B3 (encoded by a3b3) and encodes a 179 amino acid region from the 6^{th} and 7^{th} Ig domains of CEA (Oikawa et al., Biochem Biophys Res Commun 1987; 142(2): 511-8; Hefta et al., Cancer Res 1992; 52(20): 5647-55.; Zaremba et al., Cancer Res 1997; 57(20): 4570-7; Nukaya et al., Int J Cancer 1999; 80(1): 92-7; Gu et al., Gastroenterology 2020; 158(1): 238-52); the MUC1 domain is comprised of 140 amino acids, representing 7 repeat regions from the human MUC1 protein (Engelmann et al., J Biol Chem 2001; 276(30): 27764-9; Soares et al., J Immunol 2001; 166(11): 6555-63; Scheikl-Gatard et al., J Transl Med 2017; 15(1): 154; Guan et al., Bioconjug Chem 1998; 9(4): 451-8). The DNA sequence of the LOA master gene cassette is described in SEQ ID No:11 and the encoded fusion protein in SEQ ID No:12; the ClyA^{I198N} master gene cassette is described in SEQ ID No:19 and the encoded fusion protein in SEQ ID No:20.

These master gene cassettes are both designed such that cleavage of either cassette with the restriction enzymes XbaI and AvrII, followed by relegation, will result in a truncated gene encoding a fusion protein containing only the surface expression domain, linker, the A3B3 domain, and a final linker (SEQ ID NO:13 and 14 for LOA as an example). Similarly, cleavage of either cassette with NheI and Xbal, followed by relegation, will result in a truncated gene encoding a fusion protein containing only the surface expression domain and the MUC1 domain, separated by a single linker sequence (SEQ ID NO:17 and 18 for LOA as an example). These master gene cassettes were designed in this manner in order to quickly generate isogenic genetic constructs that could be used in isogenic carrier vaccines to immunize mice and determine the contribution of either A3B3, MUC1, or both, to the elicitation of a cancer-specific immune response.

### Example 6: Construction of ΔƒliC::lpxE Strains

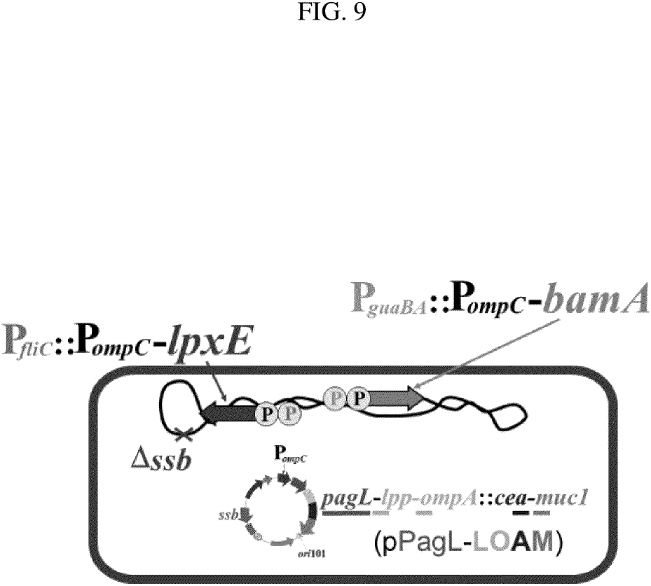

As demonstrated in our data, we have now successfully constructed a hypervesiculation reagent strain derived from the attenuated S. Typhi strain CVD 910, in which the foreign outer membrane protein target vaccine antigens AbOmpA and AbBamA from A. baumannii have been co-expressed on the surface and can now be exported (through the action of PagL) from the reagent strain via rOMVs for use as parenteral vaccines. We have demonstrated that these vesicles are reduced in reactogenicity due to the enzymatic activity of PagL which deacylates lipid A and reduces reactogenicity in vitro by approximately 10-fold. However, it has been previously reported that purified rOMVs may have varying but significant amounts of flagellin adsorbed to the surface which is an agonist for TLR5. Since we contemplate the use of purified rOMVs as vaccines administered by intramuscular injection into humans, unacceptable reactogenicity elicited by both TLR4 and TLR5 agonists must be minimized while still preserving optimal immunogenicity and protective efficacy (Liu, Q. et al., Sci. Rep. 6, 34776, doi:10.1038/srep34776 (2016)). Therefore, to further improve the clinical acceptability and purity of our rOMV-based vaccines, we reduced both TLR4- and TLR5-mediated reactogenicity by replacing chromosomally encoded FliC (a TLR5 agonist; GenBank locus #AE014613) with lpxE from Francisella novicida (lpxE^{Fn} ) (SEQ ID NO:26) (Zhao, J. & Raetz, C. R., Mol. Microbiol. 78, 820-836, doi:10.1111/j.1365-2958.2010.07305.x (2010); Zhao, J. et al., mBio 10, doi:10.1128/mBio.00886-19 (2019)). LpxE (SEQ ID NO:27) is a lipid A 1-phosphatase which dephosphorylates lipid A to produce a less reactogenic monophosphoryl species (FIG. 8); through co-expression of PagL (which deacylates lipid A while promoting hypervesiculation) rOMVs are produced containing pentaacylmonophosphoryl-lipid A with significantly reduced TLR 4 and TLR5 activity. For strain engineering, we used the well-established lambda Red site-specific chromosomal recombination system that has been used thus far for all of the strain constructions reported herein (Datsenko et al, Proc.Natl.Acad.Sci. U.S.A. 97, 6640-6645 (2000).). An isogenic set of strains were constructed, as listed in Table 1. A low copy plasmid expressing designated pPagL-LOAM expressing both PagL and the Lpp-OmpA-A3B3-MUC1 target CRC fusion protein was then introduced into these strains. The final strain CVD 910ΔguaBA::P_{ompC}bamA^{Ab}ΔƒliC::_{PompC}-lpxE^{Fn}(pPagL-LOAM) is graphically depicted in FIG. 9.

TABLE 1

| | |
|---|---|
| Isogenic Strain | Expected TLR Activation Level |
| CVD 910ΔguaBA::P_{ompC}-bamA^{Ab} | ↑TLR4, ↑TLR5 |
| CVD 910ΔguaBA::P_{ompC}-bamA^{Ab}(pPagL-LOAM) | ↓TLR4, ↓TLR5 |
| CVD 910ΔguaBA::P _{ompC}-bamA^{Ab}ΔƒliC | ↓TLR5 |
| CVD 910ΔguaBA::P _{ompC}-bamA^{Ab}ΔƒliC (pPagL-LOAM) | ↓TLR4, ↓TLR5 |
| CVD 910ΔguaBA::P _{ompC}-bamA^{Ab}ΔƒliC::P _{ompC}-lpxE^{Fn} | ↓TLR4, ↓TLR5 |
| CVD 910ΔguaBA::P _{ompC}-bamA^{A}^{b}ΔƒliC::P _{ompC}-lpxE^{Fn}(pPagL-LOAM) | ↓↓TLR4, ↓TLR5 |

### Example 7: Expression of CRC Fusion Protein in the 6 New Strains

Vesicles were purified from liquid cultures of the isogenic strains listed in Table 1 by low-speed centrifugation and filtration of supernatants through a 0.2 µm filter to remove bacterial cells and debris, followed by high-speed ultracentrifugation to pellet rOMVs; pellets were resuspended in PBS. The concentration of rOMVs was rigorously determined using a 3-Deoxy-D-manno-Octulosonic Acid (KDO) assay as prescribed by R.E.W. Hanock (http://cmdr.ubc.ca/bobh/method/kdo-assay/). 0.5 µg of each purified rOMV were then analyzed by Coomassie Brilliant Blue staining and western immunoblot analysis. As shown in FIG. 10A, multiple bands are detected in the Coomassie-stained samples, as would be expected with outer membrane vesicles containing the various proteins and lipoproteins found in the outer membrane of S. Typhi. Of note, a strong band migrating at approximately 50 kDa in lanes 2 and 3 of panel A disappears from lanes 4-7; this is consistent with the known molecular weight of ~50 kDa for the flagellin FliC, which was deleted from the strains in lanes 4-7. As shown in FIG. 10B, the CRC fusion protein is clearly detected in lanes 3, 5, and 7 as expected, but not as a single band. Given that the A3B3 domain of CEA contains 2 disulfide bridges in the expressed domain of the fusion protein (FIG. 10C), we hypothesized that the faster running species running below the strong top band are additional species containing one or more of the expected disulfide bridges. Even though the gel was run under reducing conditions with 200 mM dithiothreitol (DTT), disulfide bridges apparently still formed.

### Example 8 Immunogenicity of rOMVs in BALB/c Mice Receiving a Single Parenteral Dose

We have engineered a synthetic gene encoding a CRC-targeted fusion protein in which the A3B3 Ig-like domain from CEA was fused to a subdomain comprised of seven 20-residue repeats from the VNTR domain of MUC1. Since the domains used in this construction are normally expressed on the surface of gastrointestinal epithelial cells, it was necessary to ensure properly folded surface expression of the fusion protein in the S. Typhi carrier strain (and subsequently exported rOMVs) by genetic fusion of the A3B3-MUC1 fusion to an Lpp-OmpA surface localization peptide (Francisco et al., Proc Natl Acad Sci USA, (1992), 89:2713-7); the lpp-ompA-a3b3-muc1 gene encoding this fusion protein was then inserted into pPagL downstream of pagLv1 to create the final expression plasmid pPagL-LOAM. This plasmid was electroporated into CVD910ΔguaBA::P_{ompC}bamA^{Ab}ΔƒliC::P _{ompC}-lpxE^{Fn} (see Table 1) creating the CRC-targeting carrier strain CVD910ΔguaBA::P_{ompC}-bamA^{Ab}ΔƒliC::P_{ompC}-lpxE^{Fn}(pPagL-LOAM), capable of exporting the CRC-targeting vesicles designated rOMV^{LpxE-CRC}. We then isolated these vesicles and confirmed by western immunoblot analysis with rabbit antiserum raised against A3B3-MUC1 fusion protein that A3B3-MUC1 was strongly expressed in isolated vesicles (data not shown). However, in theory, reduction of both TLR4 and TLR5 activity in these vesicles could drastically affect the CRC-specific immune response elicited by these vesicles, so we conducted a preliminary dose-response immunogenicity experiment to assess humoral immunity against rOMV^{LpxE-CRC.}

50 BALB/c mice (6-8 week old) were randomly assorted into five groups and immunized intramuscularly with either 4.0 µg of empty unadjuvanted rOMV^{LpxE} empty vesicles as a negative control or unadjuvanted rOMV^{LpxE-CRC} in escalating doses 0.5, 1.0, 2.0, or 4.0 µg; we also administered PBS to an additional negative control group of 5 mice. The concentration of rOMVs was rigorously determined using a 3-Deoxy-D-manno-Octulosonic Acid (KDO) assay. Sera were collected on days 0 and 21, and antigen-specific serum IgG was measured in pooled sera by ELISA. As shown in Table 2, robust serum IgG titers against A3B3-MUC1 were detected after a single intramuscular dose of rOMVs, with titers appearing to peak at doses of 2 µg. We therefore chose 2 mg for further dosing experiments.

TABLE 2

CRC-specific IgG responses in BALB/c mice immunized IM with rOMVs

| rOMV | Dose | Day 0* | Day 21* |
|---|---|---|---|
| rOMV^{LpxE} | 4.0 µg | 13 | 13 |
| rOMV^{LpxE-CRC} | 0.5 µg | 13 | 11,549 |
| rOMV^{LpxE-CRC} | 1.0 µg | 13 | 20,334 |
| rOMV^{LpxE-CRC} | 2.0 µg | 13 | 36,844 |
| rOMV^{LpxE-CRC} | 4.0 µg | 13 | 38,889 |
| PBS | NA | 13 | 13 |

* mean values of pooled sera reported in EU/ml

### Example 9: Immunogenicity of rOMVs in BALB/c Mice Receiving Two Parenteral Doses Spaced 3 Weeks Apart

Although strong humoral responses were observed with rOMV^{LpxE-CRC} vesicles despite reduced TLR activation, we nonetheless wished to examine the immunogenicity of the full panel of A3B3-MUC1 expressing rOMVs with stepwise reduction of TLR activity, as summarized in Table 3. In addition to examining CRC-specific serum IgG responses, we also examined T cell responses, as judged by interferon γ (IFN- γ) ELISpot assays. We hypothesize that rOMVs present a unique opportunity for therapeutic treatment of colorectal cancer by disruption of the immunosuppressive tumor microenvironment through activation of innate responses that in turn elicit tumor-specific adaptive responses of CD8+ cytotoxic T lymphocytes.

TABLE 3

Engineered candidate carrier vaccine strains and rOMVs

| | | | |
|---|---|---|---|
| Vaccine strain (simplified nomenclature) | Genotype of candidate vaccine strain | Vesicles | Expected TLR Activation Level |
| CVD911 | CVD 910ΔguaBA::P_{ompC}-bamA^{Ab} | rOMV⁹¹¹ | ↑TLR4, ↑TLR5 |
| CVD911^{CRC} | CVD 910ΔguaBA::P _{ompC}- bamA^{Ab}(pPagL-CRC) | rOMV^{CRC} | ↓TLR4, ↑TLR5 |
| CVD911^{ΔfliC} | CVD 910ΔguaBA::P _{omPC}- bamA^{Ab}ΔfliC | rOMV^{ΔfliC} | ↑TLR4, ↓↓TLR5 |
| CVD911 ^{ΔfliC-CRC} | CVD 910ΔguaBA::P _{ompC}- bamA^{Ab}ΔfliC(pPagL-CRC) | rOMV^{ΔfliC-CRC} | ↓TLR4, ↓↓TLR5 |
| CVD911^{lpxE} | CVD 910ΔguaBA::P _{ompC}- bamA^{Ab}ΔfliC::P _{ompC}-lpxE^{Fn} | rOMV^{LpxE} | ↓TLR4, ↓↓TLR5 |
| CVD911 ^{lpxE-CRC} | CVD910ΔguaBA::P _{ompC}- bamA^{Ab} ΔfliC::P_{ompC}lpxE^{Fn}(pPacL-CRC) | rOMV ^{LpxE-CRC} | ↓↓TLR4, ↓↓TLR5 |

60 BALB/c mice (6-8 week old) were randomly assorted into six groups and immunized intramuscularly with 2.0 µg of unadjuvanted rOMV vesicles on days 0 and 21; we also administered PBS to an additional negative control group of 10 mice. Sera were collected on days 0, 20 and 35. Antigen-specific serum IgG was measured by ELISA and IFN-γ responses were determined using harvested splenocytes and a MabTech Mouse IFN-γ ELISpotPLUS HRP kit according to manufacturer’s instructions. Surprisingly, robust serum IgG titers against A3B3-MUC1 were again observed after intramuscular immunization of mice with 2 µg rOMVs, regardless of genetic manipulation of TLR activity (Table 4). Despite a high background in the ELISpot assay, IFN-γ responses against A3B3-MUC1 were clearly higher in animals receiving rOMV^{Dƒlic-CRC}, as shown in FIG. 11.

TABLE 4

CRC-specific serum IgG responses in BALB/c mice immunized IM with 2 µg of the rOMVs listed in Table 2

| Group | rOMV | Day 0* | Day 20* | Day 35* |
|---|---|---|---|---|
| A | rOMV⁹¹¹ | 13 | 19 | 25 |
| B | rOMV^{CRC} | 13 | 45,630 | 1,138,589 |
| C | rOMV^{ΔƒliC} | 13 | 13 | 25 |
| D | rOMV^{ΔƒliC-CRC} | 13 | 36,633 | 2,321,959 |
| E | rOMV^{LpxE} | 13 | 13 | 43 |
| F | rOMV ^{LpxE-CRC} | 13 | 47,002 | 1,767,484 |
| G | PBS | 13 | 13 | 13 |

* values from pooled sera reported in EU/ml

### Example 10. Immunogenicity of rOMVs in C57BL/6 Mice Receiving Two Parenteral Doses Spaced ONE Week Apart.

We anticipate conducting proof-of-concept therapeutic efficacy studies examining tumor reduction in a murine mouse model by eliciting CRC-specific immunity against our A3B3-MUC1 fusion protein. Therefore, we will require a syngeneic tumor model in which both innate and adaptive immunity are fully functional; we will therefore target tumors induced in C57BL/6 mice using a syngeneic MC38-CEA cell line purchased from Kerafast. This MC38-CEA model has been used in preclinical safety studies of a CEA vaccine antigen carrying the immunodominant T cell epitope CEA(6D) (also included in our engineered fusion protein); significant therapeutic effects were reported in an MC38-CEA mouse model which translated well to clinical trials (Crosby et al., Journal for immunotherapy of cancer, (2020), 8(2); Morse et al., J Clin Invest, (2010), 120:3234-41; Osada et al., Cancer Immunol Immunother 2012; 61(11): 1941-51).

Recent data from animal models indicates that the intrinsic size and other attendant physical characteristics of outer membrane vesicles alone can facilitate a direct therapeutic effect on tumors, independent of the immunological targeting of tumor-associated antigens, through the phenomenon of enhanced permeation and retention (EPR) that depends on the leaky vasculature of tumors (Irvine et al., Nat Rev Immunol, 2020, 20:321-34; Fang et al., Advanced drug delivery reviews, (2020), 157: 142-60; Kelly et al., Expert review of vaccines, (2019), 18: 269-80. This leaky tumor vasculature allows for passive extravasation of small nanostructures such as OMVs from the blood into tumor tissue (Fang et al., Advanced drug delivery reviews, (2020), 157: 142-60). From within the tumor tissue, these OMVs can then passively drain through the lymphatic system into regional lymph nodes, potentially encountering and activating antigen presenting cells including dendritic cells to elicit tumor-specific immunity (Fang et al., Advanced drug delivery reviews, (2020), 157: 142-60; Kelly et al., Expert review of vaccines, (2019), 18: 269-80).

To take advantage of this aspect of our rOMVs, we therefore needed to verify the humoral and cellular immunity of our rOMVs in C57BL/6 mice immunized intravenously with rOMVs. We also wished to examine the immunogenicity of a heterologous prime-boost strategy in which both rOMVs and live carrier strains are used in the vaccination regime. 35 C57BL/6 mice (6-8 week old) were randomly assorted into six groups and primed on day 0 either with unadjuvanted rOMV vesicles or live carrier vaccine; we also administered PBS to an additional negative control group of 5 mice (see Table 5, “priming dose”). All mice were boosted on day 7 with either live carrier vaccine or unadjuvanted rOMV vesicles (see Table 5, “boosting dose”). Sera were collected on days 0, 6, and 20, and splenocytes were harvested on day 21. Antigen-specific serum IgG was measured by ELISA, and IFN-γresponses were again determined using freshly harvested splenocytes in the ELISpot assay. Surprisingly, robust CRC-specific serum IgG responses were observed a mere 6 days after administration of all parenteral priming doses. Responses increased dramatically 14 days later after administration of all booster doses, regardless of route of administration or vaccine vehicle being tested.

TABLE 5

Optimization of tumor-specific humoral and cellular immune responses in C57BL/6 mice immunized by different routes

| Group | Priming dose (d0) 1 | Boosting dose (d7) | Day 0 ² (pre) | Day 6 ² (after 1 dose) | Day 20 ² (after 2 doses) |
|---|---|---|---|---|---|
| A | rOMV^{ΔfliC-CRC}; IV | CVD911^{ΔfliC-CRC}; IN | 13 | 8,101 | 61,264 |
| B | CVD911^{ΔfliC}^{-CRC}; IN | rOMV^{ΔfliC-CRC}; IV | 13 | 84 | 15,573 |
| C | rOMV^{ΔfliC-CRC}; IV | rOMV^{ΔfliC-CRC}; IV | 13 | 10,019 | 236,486 |
| D | rOMV^{ΔfliC-CRC}; IV | rOMV^{ΔlpxE-CRC}; IV | 13 | 9,109 | 165,752 |
| E | rOMV^{ΔfliC-CRC}; IV | rOMV^{ΔfliC-CRC}; IM | 13 | 8,709 | 112,675 |
| F | rOMV^{ΔfliC-CRC}; IM | rOMV^{ΔfliC-CRC}; IM | 13 | 7,561 | 126,373 |
| G | PBS | PBS | 13 | 13 | 13 |

¹ IV dose of rOMV was 0.25 µg; IM dose of rOMV was 2.0 µg; IN dose of live vaccine strain was ~1 × 10⁹ CFU
² values of pooled sera reported in EU/ml

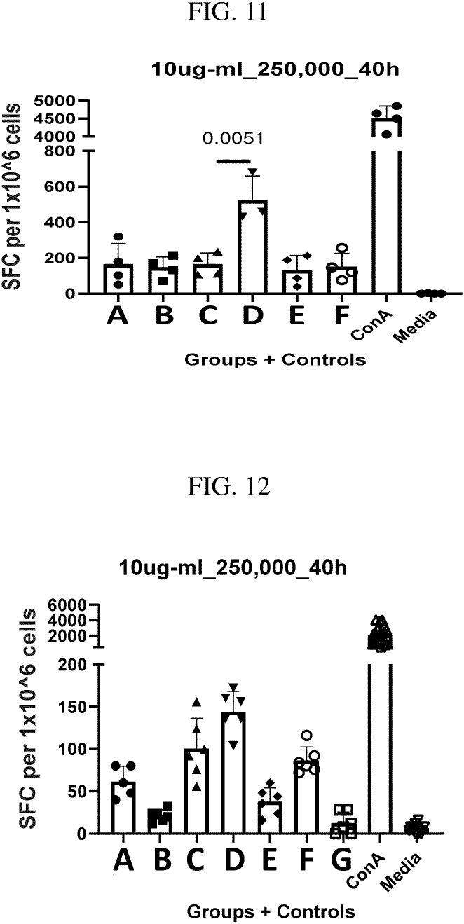

For IFN-γ ELISpot assays, splenocytes were plated in mouse IFN-γ ELISpot plates (250,000 cells/well; six replicates per rOMV vaccine) and stimulated with either (1) A3B3-MUC1 (10 µg/ml) (2) concanavalin A (4 µg/ml - positive control) or (3) media (negative control). The cells were incubated for 40 hours (37° C., 5% CO₂), then washed, and an anti-IFN-γ (biotinylated) antibody was added. Following 1h incubation, the plates were washed, streptavidin conjugated to horse radish peroxidase (SA-HRP) was added (1h), and the plates were washed again. Spot forming cells (SFC) were developed using tetramethylbenzidine (TMB) HRP substrate (7-8 min). Plates were scanned, and the spots were counted with an ELISpot Immunospot® reader (Cellular Technology). Data were analyzed with Immunospot® version 7.0 software. The total of SFC/well were reported as SFC/10⁶ splenocytes, and results are reported in FIG. 12.

Both the humoral and cellular responses are lower for this experiment versus those reported in Example 9, likely due to the shortened timeframe between priming and boosting (7 days for Example 10 versus 21 days for Example 9) and the shortened timeframe for the duration of the experiment (21 days for Example 10 versus 35 days for Example 9); a closer spacing for the prime-boosting protocol used in Example 10 was adopted based on the work of Kim et al and Cheng et al, in which both groups demonstrated therapeutic tumor reduction in mice receiving closely spaced IV injections of OMVs (Kim et al., Nature communications, (2017), 8:626); Cheng et al., Nature communications, (2021), 12: 2041). Despite these modifications, all IFN-γ responses against A3B3-MUC1 reported in FIG. 12 were statistically significant (p≤ 0.05 versus the PBS control Group G) and closely corresponded to A3B3-MUC1-specific antibody responses (Table 5). The highest IFN-γ responses were elicited in animals receiving homologous dosing with rOMVs (i.e. groups C and D immunized with 0.25 µg of unadjuvanted rOMVs administered IV, or group F immunized with 2.0 µg of unadjuvanted rOMVs administered IM). Heterologous prime-boost dosing either with rOMVs and live attenuated carrier strains (Groups A and B) or with the same rOMV administered by different routes (Group E, IV versus IM) elicited the lowest CRC-specific antibody and cellular responses.

Taken together, results from Examples 8-10 firmly establish the feasibility of engineering an attenuated S. Typhi-based carrier strain that can efficiently express and deliver targeted colorectal A3B3-MUC1 proteins to elicit antigen-specific immunity. We have successfully engineered a unique PagL-mediated outer membrane vesicle antigen delivery platform in which the efficiency of rOMV export and isolation is enhanced by the over-expression of the outer membrane folding protein BamA. We have purified these rOMVs and demonstrated both humoral and cellular antigen-specific immunity against our CRC targeted A3B3-MUC1 fusion protein. These data unambiguously demonstrate that intravenous administration into C57BL/6 mice of unadjuvanted rOMVs induces excellent CRC-specific immunity, whereas heterologous prime-boosting with rOMVs and live carrier strains induced the lowest immune responses. In a preferred embodiment, we will pursue further development of purified rOMVs intended for intravenous administration into humans as a therapeutic immunological intervention against primary tumor progression and prevention of metastasis to the liver; we will exploit engineered rOMVs in which strong innate stimulation via TLR 4 and 5 agonists is reduced to improve the safety and clinical acceptability of intravenously administered rOMVs, by avoiding septic shock or induction of cytokine storm responses in tumor patients.

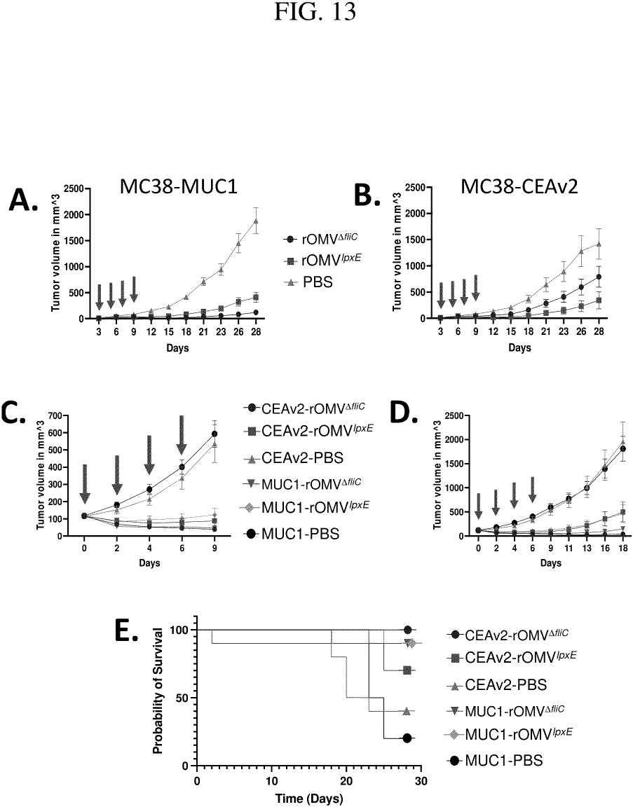

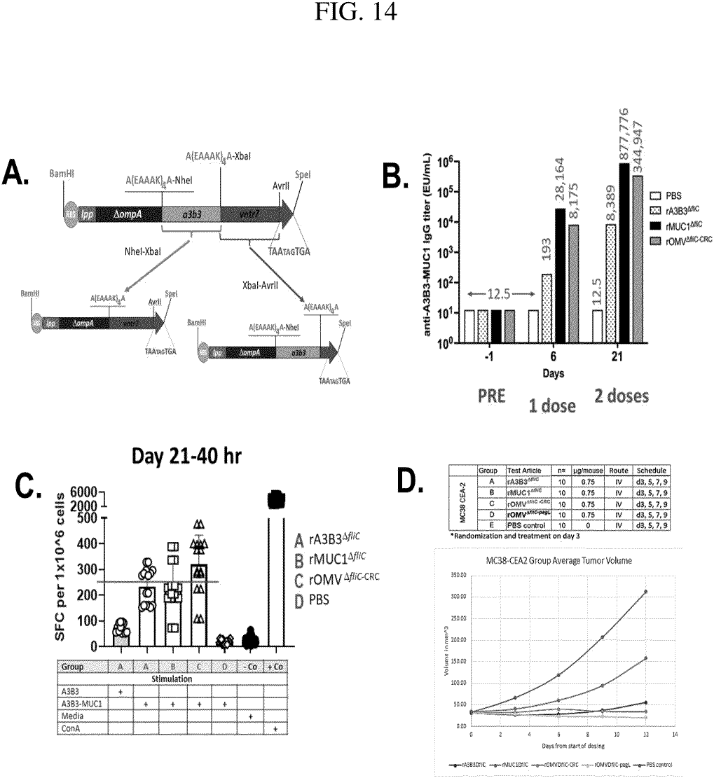

Example 11. Therapeutic efficacy of rOMVs expressing a CEA-MUC1 fusion protein in a syngeneic C57BL/6 mouse tumor challenge model. Despite the re-engineering of the rOMVs to reduce TLR4 and TLR5 activity, these modified vesicles still maintained robust immunogenicity when tested in C57BL/6 mice, with excellent serum IgG responses against our CEA-MUC1 fusion protein, as well as excellent antigen-specific cellular responses as judged by INFγ ELISPOT assays. Encouraged by these very strong humoral and cellular responses, we conducted an initial therapeutic efficacy study using a syngeneic C57BL/6 mouse model implanted with MC38 murine tumor cells expressing either CEA or MUC1. Mice (10 mice per group) were implanted subcutaneously with 300,000 tumor cells on day 1 and then treated intravenously with 0.75 µg of vesicles (based on quantitation of LPS concentration using a KDO assay) on days 3, 5, 7, and 9. Progression of tumors was monitored by calculating tumor volumes through day 28. As shown in FIGS. 13A and B, all groups treated intravenously with rOMVs experienced reductions in tumor volumes. When examining individual mice, 50% of mice challenged with MC38-MUC1experienced total remission of tumors when treated with rOMV^{ΔfliC-CRC} and 20% total remission in mice treated with rOMV^{lpxE-}CRC; for mice challenged with MC38-CEAv2, 50% experienced total remission when treated with rOMV^{lpxE-CRC} while 30% total remission was observed in mice treated with rOMV^{ΔfliC-CRC}. Having demonstrated an ability of our rOMVs to significantly block the progression of a nascent tumor, we then investigated their ability to therapeutically treat an existing tumor. C57BL/6 mice were again implanted with 300,000 tumor cells on day 1 and tumors allowed to progress to average tumor volumes between 115 and 120 mm³. Mice were then treated intravenously with 0.75 µg of vesicles on days 0, 2, 4, and 6. Due to the progression of tumors leading to the need for compassionate euthanasia, tumor volumes were only recorded through day 18 (FIGS. 13C and D); mortality was then recorded through day 28 (FIG. 13E). As with the previous experiment, we observed significant reductions in tumor volumes in all groups intravenously treated with vesicles. As shown in FIG. 13C, initial tumor volumes began to recede after the first IV dose and remained significantly lower than untreated PBS control groups for all experimental groups through day 18 (FIG. 13D). Survival data reported in FIG. 13E demonstrate ≥90% survival in groups treated with rOMV^{ΔfliC-CRC} regardless of the challenge tumor cell line used; although total remission was not observed in any individual mice for this experiment, we believe these data support the currently prevailing strategy in oncology whereby initiating treatment early (as modeled in experiments summarized in FIGS. 13A and B) can lead to more successful therapeutic outcomes in patients. Taken together, the data from these two challenge studies suggest that genetic modification of lipid A does not interfere with the therapeutic efficacy of our rOMVs.

### Example 12. Therapeutic Efficacy of rOMVs Expressing Individual CRC Targeted Proteins Versus the CRC Fusion Protein in a Syngeneic C57BL/6 Mouse Tumor Challenge Model

We were curious as to why the efficacy against MC38-CEAv2 challenges was less than that observed when challenging with MC38-MUC1 cells. We therefore elected to construct isogenic constructs in which expression plasmids expressing the CRC fusion protein were deleted of either the A3B3 domain or the MUC1 domain, as shown in FIG. 14A. Since we were only interested here in assessing the immunogenicity of individual domains versus the fusion protein, we focused only on modification of the rOMV^{ΔfliC-CRC} construct expressing the full-length A3B3-MUC1 fusion protein. Deletion of the A3B3 domain created rMUC1^{ΔfliC}, expressing only the 7 repeats of the human MUC1 protein (see FIG. 13A); likewise, deletion of the MUC1 domain created rA3B3^{ΔfliC}, expressing only the A3B3 domain of the human CEA protein. These re-engineered plasmids were then re-introduced by standard electroporation back into the carrier strain CVD911 for expression and purification of the therapeutic candidate rOMVs. Using these re-engineered rOMVs and comparing to unmodified rOMV^{ΔfliC-CRC}, we immunized mice as described in Table 5 above. 35 C57BL/6 mice (6-8 week old) were randomly assorted into three groups and primed intravenously on day 0 with 0.25 µg of unadjuvanted rOMV vesicles; we also administered PBS to an additional negative control group of 5 mice. All mice were intravenously boosted on day 7 with 0.25 µg of unadjuvanted rOMV vesicles or PBS. Sera were collected on days 0, 6, and 21, and splenocytes were harvested on day 21 for ELISPOT analysis; results are reported in FIGS. 14B and C respectively. These data suggest that MUC1 is strongly immunogenic while A3B3 is less immunogenic when examining tumor-specific serum IgG responses (FIG. 14B); this difference in immunogenicity is less apparent when measuring tumor-specific T cell responses as judged by the ELISPOT assay (FIG. 14C). Having demonstrated that both independent domains elicited humoral and cellular tumor-specific responses, we then conducted another syngeneic tumor challenge experiment in which C57BL/6 mice were implanted on day 0 with 300,000 MC38-CEAv2 cells and then treated intravenously with 0.75 µg on days 3, 5, 7, and 9 with either rA3B3^{ΔfliC}, rMUC1^{ΔfliC}, rOMV^{ΔfliC-CRC}, or empty rOMVΔ^{fliC-}^{pagL} vesicles. We only challenged with MC38-CEAv2 cells in an attempt to distinguish antigen-specific protection against CEAv2. We hypothesized that if MUC1 is immunodominant, we should observe therapeutic efficacy against the CEAv2 challenge from rA3B3^{ΔfliC} and rOMV^{ΔfliC-CRC}, while reduced efficacy would be seen with rMUC1^{ΔfliC} and no efficacy from empty vesicles. As shown in FIG. 14D, we continued to see excellent therapeutic efficacy against MC38-CEAv2 conferred by rOMV^{ΔfliC-CRC}, and reduced efficacy conferred by rMUC1^{ΔfliC}against challenge with MC38-CEAv2. Surprisingly, excellent efficacy was elicited by rA3B3^{ΔfliC}, a response not predicted based on humoral and cellular responses (FIGS. 14B and C). Most surprising, empty vesicles also conferred excellent efficacy against challenge with MC38-CEAv2, despite expressing no tumor-specific antigen(s) on the empty rOMV surface. However, the EPR effect should also have been as effective with rA3B3^{ΔfliC} and rMUC1^{ΔfliC}vesicles but was not. Although we do not have a clear explanation of this discrepancy, we suspect that rOMV surface charge is involved. It is possible that native unmodified surface charge allows tumor penetration by empty vesicles whereas introduction of either the A3B3 domain or the MUC1 domain alone perturbs surface charge and diminishes tumor penetration by the EPR effect, without affecting the induction of tumor-specific immunity that results in at least some efficacy relative to PBS controls. By this reasoning, the A3B3-MUC1 fusion protein apparently does not interfere either with the EPR effect or induction of tumor-specific immunity.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

We claim:

1. A live Salmonella Typhi vector that has been engineered to express:
a one or more cancer antigens;
b an outer membrane folding protein BamA or a fragment or variant thereof; and
c a lipid A deacylase PagL or a fragment or variant thereof,
wherein the Salmonella Typhi vector is capable of delivering the antigen to a mucosal tissue via an outer membrane vesicle when administered to a subject.

2. The Salmonella Typhi vector of claim 1, wherein the antigen is an outer membrane protein.

3. The Salmonella Typhi vector of claim 1, wherein the antigen is encoded by a nucleic acid that is chromosomally integrated in S. Typhi.

4. The Salmonella Typhi vector of claim 1, wherein the antigen is expressed from a plasmid.

5. The Salmonella Typhi vector of claim 1, wherein the Salmonella Typhi vector comprises a deletion in guaBA and htrA.

6. The Salmonella Typhi vector of claim 1, wherein the antigen is inserted into an S. Typhi locus selected from the group consisting of guaBA, rpoS, htrA, ssb, and combinations thereof.

7. (canceled)

8. The Salmonella Typhi vector of any of claim 1, wherein the S. Typhi overexpresses a cytolysin A (ClyA) protein to facilitate outer membrane vesicle formation.

9. The Salmonella Typhi vector of claim 8, wherein the ClyA is mutated to reduce hemolytic activity of ClyA.

10. The Salmonella Typhi vector of claim 9, wherein the ClyA mutant is selected from the group consisting of ClyA I198N, ClyA A199D, ClyA E204K, ClyA C285W and combinations thereof.

11. (canceled)

12. (canceled)

13. The Salmonella Typhi vector of any of claim 1, wherein the BamA is from Acinetobacter baumannii.

14. The Salmonella Typhi vector of any of claim 1, wherein the BamA amino acid sequence comprises SEQ ID NO:8.

15. The Salmonella Typhi vector of claim 14, wherein the bamA gene encoding BamA protein is integrated into the genome of Salmonella Typhi.

16. The Salmonella Typhi vector of claim 15, wherein bamA is integrated into the guaBA locus of Salmonella Typhi.

17. The Salmonella Typhi vector of claim 13, wherein bamA is expressed by an inducible promoter.

18. The Salmonella Typhi vector of claim 17, wherein the inducible promoter is osmotically controlled.

19. The Salmonella Typhi vector of claim 18, wherein the osmotically controlled inducible promoter is a promoter of Outer Membrane Protein C (ompC) gene.

20. The Salmonella Typhi vector of claim 19, wherein the promoter of Outer Membrane Protein C (ompC) gene comprises SEQ ID NO:9.

21. The Salmonella Typhi vector of claim 1, wherein the pagL gene encoding PagL is integrated into the genome of Salmonella Typhi.

22. The Salmonella Typhi vector of claim 1, wherein pagL is expressed from a plasmid.

23. The Salmonella Typhi vector of claim 22, wherein the plasmid expressing PagL is a low-copy-number expression plasmid.

24. The Salmonella Typhi vector of claim 1, wherein expression of pagL is controlled by an inducible promoter.

25. The Salmonella Typhi vector of claim 22, wherein the plasmid has a non-antibiotic-based plasmid selection system.

26. The Salmonella Typhi vector of claim 25, wherein the plasmid expresses a gene that is essential for the growth of S. Typhi and has been chromosomally mutated in S. Typhi.

27. The Salmonella Typhi vector of claim 26, wherein the gene encodes single stranded binding protein (SSB).

28. (canceled)

29. (canceled)

30. The Salmonella Typhi vector of claim 22, wherein the plasmid further encodes and expresses the antigen.

31. The Salmonella Typhi vector of claim 1, wherein the PagL amino acid sequence is selected from SEQ ID NO:2 and SEQ ID NO:4.

32. (canceled)

33. (canceled)

34. (canceled)

35. (canceled)

36. The Salmonella Typhi vector of claim 1, wherein the cancer is a colon cancer antigen.

37. The Salmonella Typhi vector of claim 1, wherein the Salmonella Typhi vector comprises two cancer antigens.

38. The Salmonella Typhi vector of claim 1, wherein the cancer antigen is fused to a polypeptide to facilitate surface presentation of the antigen.

39. The Salmonella Typhi vector of claim 1, wherein the cancer antigen is fused to a chimeric Lpp-OmpA surface display polypeptide.

40. (canceled)

41. The Salmonella Typhi vector of claim 36, wherein the colon cancer antigens are selected from CEA or an antigenic fragment thereof, MUC1 or an antigenic fragment thereof and a combination thereof.

42-157. (canceled)


SEQUENCE LISTING (31 sequences)

SEQ ID NO: 1 - DNA, 650 bp, synthetic construct
  misc_feature 1..650: note = pagL
  source 1..650: mol_type = other DNA
ggatccaattgaaaataaggaggaaaaaatgactagtatgaagcgcatctttatttatctgttacttccgtgtgcattcgcatgttctgctaatgataatgttttttttggcaagggcaacaagcatcagatctcttttgctgcgggagaaagtataagaagaggaggggttgagcacttatatacggcttttctgacatacagtgaacccagcgattttttctttttacaggcaagaaataatctggagttaggaggatttaaggctaagggtagcgatgattgcagtaaacattctggcagcgttccctgtaataaatataaccagggcgtattgggtatctcgaaggatgtggcgctggttcatttcgctggtatctataccggtattggtctgggggcttatataaaatctaagtcgcgagatgatatgcgtgtcaattctgcatttacctttggagaaaaagcgtttcttggctggaactttggggctttttctacagaagcttatatccggcatttctcgaatggatcacttacggataaaaattcagggcataattttgtaggtgcttcaattagttataatttctgataatagttgataacctaggccgcctaatgagcgggcttttttttctcgggctagc

SEQ ID NO: 2 - protein, 188 aa, Salmonella enterica
MTSMKRIFIYLLLPCAFACSANDNVFFGKGNKHQISFAAGESIRRGGVEHLYTAFLTYSEPSDFFFLQARNNLELGGFKAKGSDDCSKHSGSVPCNKYNQGVLGISKDVALVHFAGIYTGIGLGAYIKSKSRDDMRVNSAFTFGEKAFLGWNFGAFSTEAYIRHFSNGSLTDKNSGHNFVGASISYNF

SEQ ID NO: 3 - DNA, 647 bp, synthetic construct
  misc_feature 1..647: note = pagL
  source 1..647: mol_type = other DNA
ggatccaattgaaaataaggaggaaaaagtgtatatgaagcgcatctttatttatctgttacttccgtgtgcattcgcatgttctgctaatgataatgttttttttggcaagggcaacaagcatcagatctcttttgctgcgggagaaagtataagaagaggaggggttgagcacttatatacggcttttctgacatacagtgaacccagcgattttttctttttacaggcaagaaataatctggagttaggaggatttaaggctaagggtagcgatgattgcagtaaacattctggcagcgttccctgtaataaatataaccagggcgtattgggtatctcgaaggatgtggcgctggttcatttcgctggtatctataccggtattggtctgggggcttatataaaatctaagtcgcgagatgatatgcgtgtcaattctgcatttacctttggagaaaaagcgtttcttggctggaactttggggctttttctacagaagcttatatccggcatttctcgaatggatcacttacggataaaaattcagggcataattttgtaggtgcttcaattagttataatttctgataatagttgataacctaggccgcctaatgagcgggcttttttttctcgggctagc

SEQ ID NO: 4 - protein, 187 aa, Salmonella enterica
MYMKRIFIYLLLPCAFACSANDNVFFGKGNKHQISFAAGESIRRGGVEHLYTAFLTYSEPSDFFFLQARNNLELGGFKAKGSDDCSKHSGSVPCNKYNQGVLGISKDVALVHFAGIYTGIGLGAYIKSKSRDDMRVNSAFTFGEKAFLGWNFGAFSTEAYIRHFSNGSLTDKNSGHNFVGASISYNF

SEQ ID NO: 5 - DNA, 655 bp, synthetic construct
  misc_feature 1..655: note = pagL
  source 1..655: mol_type = other DNA
ggatccaattgctattgacattgaaatggtggtggaatgtatatgaagagaatatttatatatctattattaccttgtgcattcgcatgttctgctaatgataatgttttttttggcaagggcaacaagcatcagatctcttttgctgcgggagaaagtataagaagaggaggggttgagcacttatatacggcttttctgacatacagtgaacccagcgattttttctttttacaggcaagaaataatctggagttaggaggatttaaggctaagggtagcgatgattgcagtaaacattctggcagcgttccctgtaataaatataaccagggcgtattgggtatctcgaaggatgtggcgctggttcatttcgctggtatctataccggtattggtctgggggcttatataaaatctaagtcgcgagatgatatgcgtgtcaattctgcatttacctttggagaaaaagcgtttcttggctggaactttggggctttttctacagaagcttatatccggcatttctcgaatggatcacttacggataaaaattcagggcataattttgtaggtgcttcaattagttataatttctgataatagttgataacctaggccgcctaatgagcgggcttttttttctcgggctagc

SEQ ID NO: 6 - DNA, 6909 bp, synthetic construct
  misc_feature 1..6909: note = pagL
  source 1..6909: mol_type = other DNA
gaattctgtggtagcacagaataatgaaaagtgtgtaaagaagggtaaaaaaaaccgaatgcgaggcatccggttgaaataggggtaaacagacattcagaaatgaatgacggtaataaataaagttaatgatgatagcgggagttattctagttgcgagtgaaggttttgttttgacattcagtgctgtcaaatacttaagaataagttattgattttaaccttgaattattattgcttgatgttaggtgcttatttcgccattccgcaataatcttaaaaagttcccttgcatttacattttgaaacatctatagcgataaatgaaacatcttaaaagttttagtatcatattcgtgttggattattctgcatttttggggagaatggacttgccgactgattaatgagggttaatcagtatgcagtggcataaaaaagcaaataaaggcatataacagatcgatcttaaacatccacaggaggatgggatccaattgaaaataaggaggaaaaaatgactagtatgaagcgcatctttatttatctgttacttccgtgtgcattcgcatgttctgctaatgataatgttttttttggcaagggcaacaagcatcagatctcttttgctgcgggagaaagtataagaagaggaggggttgagcacttatatacggcttttctgacatacagtgaacccagcgattttttctttttacaggcaagaaataatctggagttaggaggatttaaggctaagggtagcgatgattgcagtaaacattctggcagcgttccctgtaataaatataaccagggcgtattgggtatctcgaaggatgtggcgctggttcatttcgctggtatctataccggtattggtctgggggcttatataaaatctaagtcgcgagatgatatgcgtgtcaattctgcatttacctttggagaaaaagcgtttcttggctggaactttggggctttttctacagaagcttatatccggcatttctcgaatggatcacttacggataaaaattcagggcataattttgtaggtgcttcaattagttataatttctgataatagttgataacctaggccgcctaatgagcgggcttttttttctcgggctagctgataacctagcccgcctaatgagcgggcttttttttctcggcctaggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcaaccggatctaaaacactaggcccaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagctggatctaaaacactagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagccgtggcaattccggttcgctgctagacaacatcagcaaggagaaaggggctaccggcgaaccagcagcccctttataaaggcgcttcagtagtcagaccagcatcagtcctgaaaaggcgggcctgcgcccgcctccaggttgctacttaccggattcgtaagccatgaaagccgccacctccctgtgtccgtctctgtaacgaatctcgcacagcgattttcgtgtcagataagtgaatatcaacagtgtgagacacacgatcaacacacaccagacaagggaacttcgtggtagtttcatggccttcttctccttgcgcaaagcgcggtaagaggctatcctgatgtggactagacatagggatgcctcgtggtggttaatgaaaattaacttactacggggctatcttctttctgccacacaacacggcaacaaaccaccttcacgtcatgaggcagaaagcctcaagcgccgggcacatcatagcccatatacctgcacgctgaccacactcactttccctgaaaataatccgctcattcagaccgttcacgggaaatccgtgtgattgttgccgcatcacgctgcctcccggagtttgtctcgagcacttttgttacccgccaaacaaaacccaaaaacaacccatacccaacccaataaaacaccaaaacaagacaaataatcattgattgatggttgaaatggggtaaacttgacaaacaaacccacttaaaacccaaaacatacccaaacacacaccaaaaaaacaccataaggagttttataaatgttggtattcattgatgacggttcaacaaacatcaaactacagtggcaggaaagcgacggaacaattaaacagcacattagcccgaacagcttcaaacgcgagtgggcagtctcttttggtgataaaaaggtctttaactacacactgaacggcgaacagtattcatttgatccaatcagcccggatgctgtagtcacaaccaatatcgcatggcaatacagcgacgttaatgtcgttgcagtgcatcacgccttactgaccagtggtctgccggtaagcgaagtggatattgtttgcacacttcctctgacagagtattacgacagaaataaccaacccaatacggaaaatattgagcgtaagaaagcaaacttccggaaaaaaattacattaaatggcggggatacattcacaataaaagatgtaaaagtcatgcctgaatctataccggcaggttatgaagttctacaagaactggatgagttagattctttattaattatagatctcgggggcaccacattagatatttctcaggtaatggggaaattatcggggatcagtaaaatatacggagactcatctcttggtgtctctctggttacatctgcagtaaaagatgccctttctcttgcgagaacaaaaggaagtagctatcttgctgacgatataatcattcacagaaaagataataactatctgaagcaacgaattaatgatgagaacaaaatatcaatagtcaccgaagcaatgaatgaagcacttcgtaaacttgagcaacgtgtattaaatacgctcaatgaattttctggttatactcatgttatggttataggcggtggcgcagaattaatatgcgatgcagtaaaaaaacacacacagattcgtgatgaacgttttttcaaaaccaataactctcaatatgatttagttaacggtatgtatctcataggtaattaatgatggacaagcgcagaaccattgccttcaaactaaatccagatgtaaatcaaacagataaaattgtttgtgatacactggacagtatcccgcaaggggaacgaagccgccttaaccgggccgcactgacggcaggtctggccttatacagacaagatccccggacccctttccttttatgtgagctgctgacgaaagaaaccacattttcagatatcgtgaatatattgagatcgctatttccaaaagagatggccgattttaattcttcaatagtcactcaatcctcttcacaacaagagcaaaaaagtgatgaagagaccaaaaaaaatgcgatgaagctaataaattaattcaattattattgagttccctttatccactatcaggctggataaagggaactcaatcaagttattttcttaccagtcattacataatcgttattatgaaataatcgtttgcactgtctctgttattcaggcaatttcaataaaggcacttgctcacgctctgtcattttctgaaactcttcatgctg

SEQ ID NO: 7 - DNA, 3080 bp, synthetic construct
  misc_feature 1..3080: note = PompC-bamA (in pSEC10 vector)
  source 1..3080: mol_type = other DNA
gaattctgtggtagcacagaataatgaaaagtgtgtaaagaagggtaaaaaaaaccgaatgcgaggcatccggttgaaataggggtaaacagacattcagaaatgaatgacggtaataaataaagttaatgatgatagcgggagttattctagttgcgagtgaaggttttgttttgacattcagtgctgtcaaatacttaagaataagttattgattttaaccttgaattattattgcttgatgttaggtgcttatttcgccattccgcaataatcttaaaaagttcccttgcatttacattttgaaacatctatagcgataaatgaaacatcttaaaagttttagtatcatattcgtgttggattattctgcatttttggggagaatggacttgccgactgattaatgagggttaatcagtatgcagtggcataaaaaagcaaataaaggcatataacagatcgatcttaaacatccacaggaggatgggatccgctagcaaaataaggaggaaaaagtgcggcacacacattttttaatgcctttggcacttgttagcgctatggcagcggtacaacaagcatatgcagctgatgatttcgttgttagagatattcgtgtaaacgggttggtccgtcttacccctgcaaatgtttataccatgttgccaatcaacagtggcgatcgcgttaatgaaccgatgatcgccgaagcaattcgaacattgtatgccactggactttttgatgatattaaagcatcaaaagaaaatgatactttggtttttaatgttattgagcgcccaattatttcaaagcttgaatttaagggtaataaacttattcctaaagaggctctagaacaaggcctcaaaaaaatgggtattgctgagggtgaggtttttaagaaatctgctttgcaaaccattgagacagaattagagcaacaatatactcaacaaggccgttatgatgctgatgttacagttgatacagttgctcgcccaaataaccgcgttgaattaaaaattaatttcaatgaaggaacgccagcaaaagtctttgatattaatgtaattggtaatactgtttttaaagacagtgaaattaaacaagcatttgcagttaaagaaagtggttgggcttcagtagtcacacgtaatgaccgctatgcacgtgaaaaaatggctgcaagccttgaagctttacgtgccatgtaccttaacaaaggctatattaattttaatattaataattcccaactcaatattagtgaagataaaaagcatatctttattgaggttgctgttgatgaaggtagccagtttaaatttggtcaaactaaattcttaggtgatgcgctttataagcctgaagaattacaagctttaaaaatttataaagacggtgatacctattcacaagaaaaagtaaatgcggttaagcaattacttttacgtaagtatggtaatgcaggttattactttgcagatgtaaatattgtgccgcagattaataatgaaactggcgttgttgacttaaattattatgtgaatccaggtcaacaagttaccgtacgtcgtattaattttactggtaatagcaaaacttctgatgaagtattacgtcgtgaaatgcgtcagatggaaggtgcattagcgagcaatgaaaaaattgacttatctaaagttcgtttagagcgtacaggtttctttaaaaccgttgatattaaacctgctcgtattccaaactctccagaccaagttgacttaaacgtaaatgttgaagaacaacattcaggtactactacattagcagttggttattcgcaaagtggtggtattacgttccaagcaggacttagtcaaactaactttatgggtacaggtaaccgagttgcaattgacttatctcgttctgaaactcaagactattacaacttaagtgttactgacccttactttacaattgatggtgtaagccgcggttataatgtctattatcgtaaaacgaaactgaatgatgactataacgttaacaactatgtaacggatagttttggtggtagtttaagttttggttaccctattgatgaaaatcaaagtttaagtgcttcggtgggtgttgataatactaaggtaacaacaggtgcatttgtatccacttatgtacgtgattacttattggcaaatggtgggaaaactacttctacaaatacgtactgcctcgttgatcttgttcaagatccccaaacaggattatataaatgcccagaaggtcaaacttctcaaccttatggaaatgcgtttgaaggagagttctttacttataacctgaatttgggttggtcgtacaatactttgaatagaccaatctttccaacatcaggcatgtctcaccgtgtaggtcttgaaattggtttaccgggtagtgatgttgattatcaaaaagtaacttatgatactcaagcatttttccctattggaagtaccggctttgtacttcgtggttatggtaagcttgggtatggtaatgatttaccattctataagaacttctatgcaggtggttatggctctgtacgtggttatgataacagtactttagggcctaaatatgccagtgtaaatctacaagaagagaaaaaaaatgactcttctccagaagaagtgggtggtaatgctctagtgcagttcggcacagaattggttttaccaatgccatttaaaggtgactggactcgccaagtacgccctgttctatttgctgaaggtggtcaagtatttgataccaagtgcgacgttcgatcttattctatgattatgaatggtcagcaaatttctgatgctaaaaaatactgtgaagataattatggatttgatttaggtaacttgcgttatagtgtgggtgtcggtgtaacctggattactatgattggtccattgtcacttagctatgcattcccgttaaatgataaaccgggtgatgaaactaaagaaattcagtttgaaattggtcgtactttctgataatagatctgcacgccgcctaatgagcgggcttttttttctcggcctagg

SEQ ID NO: 8 - protein, 836 aa, Acinetobacter baumannii
MRHTHFLMPLALVSAMAAVQQAYAADDFVVRDIRVNGLVRLTPANVYTMLPINSGDRVNEPMIAEAIRTLYATGLFDDIKASKENDTLVFNVIERPIISKLEFKGNKLIPKEALEQGLKKMGIAEGEVFKKSALQTIETELEQQYTQQGRYDADVTVDTVARPNNRVELKINFNEGTPAKVFDINVIGNTVFKDSEIKQAFAVKESGWASVVTRNDRYAREKMAASLEALRAMYLNKGYINFNINNSQLNISEDKKHIFIEVAVDEGSQFKFGQTKFLGDALYKPEELQALKIYKDGDTYSQEKVNAVKQLLLRKYGNAGYYFADVNIVPQINNETGVVDLNYYVNPGQQVTVRRINFTGNSKTSDEVLRREMRQMEGALASNEKIDLSKVRLERTGFFKTVDIKPARIPNSPDQVDLNVNVEEQHSGTTTLAVGYSQSGGITFQAGLSQTNFMGTGNRVAIDLSRSETQDYYNLSVTDPYFTIDGVSRGYNVYYRKTKLNDDYNVNNYVTDSFGGSLSFGYPIDENQSLSASVGVDNTKVTTGAFVSTYVRDYLLANGGKTTSTNTYCLVDLVQDPQTGLYKCPEGQTSQPYGNAFEGEFFTYNLNLGWSYNTLNRPIFPTSGMSHRVGLEIGLPGSDVDYQKVTYDTQAFFPIGSTGFVLRGYGKLGYGNDLPFYKNFYAGGYGSVRGYDNSTLGPKYASVNLQEEKKNDSSPEEVGGNALVQFGTELVLPMPFKGDWTRQVRPVLFAEGGQVFDTKCDVRSYSMIMNGQQISDAKKYCEDNYGFDLGNLRYSVGVGVTWITMIGPLSLSYAFPLNDKPGDETKEIQFEIGRTF

SEQ ID NO: 9 - DNA, 495 bp, synthetic construct
  misc_feature 1..495: note = PompC osmotically controlled promoter
  source 1..495: mol_type = other DNA
gaattctgtggtagcacagaataatgaaaagtgtgtaaagaagggtaaaaaaaaccgaatgcgaggcatccggttgaaataggggtaaacagacattcagaaatgaatgacggtaataaataaagttaatgatgatagcgggagttattctagttgcgagtgaaggttttgttttgacattcagtgctgtcaaatacttaagaataagttattgattttaaccttgaattattattgcttgatgttaggtgcttatttcgccattccgcaataatcttaaaaagttcccttgcatttacattttgaaacatctatagcgataaatgaaacatcttaaaagttttagtatcatattcgtgttggattattctgcatttttggggagaatggacttgccgactgattaatgagggttaatcagtatgcagtggcataaaaaagcaaataaaggcatataacagatcgatcttaaacatccacaggaggatgggatcc

SEQ ID NO: 10 - DNA, 2517 bp, Acinetobacter baumannii
  source 1..2517: mol_type = other DNA
gtgcggcacacacattttttaatgcctttggcacttgttagcgctatggcagcggtacaacaagcatatgcagctgatgatttcgttgttagagatattcgtgtaaacgggttggtccgtcttacccctgcaaatgtttataccatgttgccaatcaacagtggcgatcgcgttaatgaaccgatgatcgccgaagcaattcgaacattgtatgccactggactttttgatgatattaaagcatcaaaagaaaatgatactttggtttttaatgttattgagcgcccaattatttcaaagcttgaatttaagggtaataaacttattcctaaagaggctctagaacaaggcctcaaaaaaatgggtattgctgagggtgaggtttttaagaaatctgctttgcaaaccattgagacagaattagagcaacaatatactcaacaaggccgttatgatgctgatgttacagttgatacagttgctcgcccaaataaccgcgttgaattaaaaattaatttcaatgaaggaacgccagcaaaagtctttgatattaatgtaattggtaatactgtttttaaagacagtgaaattaaacaagcatttgcagttaaagaaagtggttgggcttcagtagtcacacgtaatgaccgctatgcacgtgaaaaaatggctgcaagccttgaagctttacgtgccatgtaccttaacaaaggctatattaattttaatattaataattcccaactcaatattagtgaagataaaaagcatatctttattgaggttgctgttgatgaaggtagccagtttaaatttggtcaaactaaattcttaggtgatgcgctttataagcctgaagaattacaagctttaaaaatttataaagacggtgatacctattcacaagaaaaagtaaatgcggttaagcaattacttttacgtaagtatggtaatgcaggttattactttgcagatgtaaatattgtgccgcagattaataatgaaactggcgttgttgacttaaattattatgtgaatccaggtcaacaagttaccgtacgtcgtattaattttactggtaatagcaaaacttctgatgaagtattacgtcgtgaaatgcgtcagatggaaggtgcattagcgagcaatgaaaaaattgacttatctaaagttcgtttagagcgtacaggtttctttaaaaccgttgatattaaacctgctcgtattccaaactctccagaccaagttgacttaaacgtaaatgttgaagaacaacattcaggtactactacattagcagttggttattcgcaaagtggtggtattacgttccaagcaggacttagtcaaactaactttatgggtacaggtaaccgagttgcaattgacttatctcgttctgaaactcaagactattacaacttaagtgttactgacccttactttacaattgatggtgtaagccgcggttataatgtctattatcgtaaaacgaaactgaatgatgactataacgttaacaactatgtaacggatagttttggtggtagtttaagttttggttaccctattgatgaaaatcaaagtttaagtgcttcggtgggtgttgataatactaaggtaacaacaggtgcatttgtatccacttatgtacgtgattacttattggcaaatggtgggaaaactacttctacaaatacgtactgcctcgttgatcttgttcaagatccccaaacaggattatataaatgcccagaaggtcaaacttctcaaccttatggaaatgcgtttgaaggagagttctttacttataacctgaatttgggttggtcgtacaatactttgaatagaccaatctttccaacatcaggcatgtctcaccgtgtaggtcttgaaattggtttaccgggtagtgatgttgattatcaaaaagtaacttatgatactcaagcatttttccctattggaagtaccggctttgtacttcgtggttatggtaagcttgggtatggtaatgatttaccattctataagaacttctatgcaggtggttatggctctgtacgtggttatgataacagtactttagggcctaaatatgccagtgtaaatctacaagaagagaaaaaaaatgactcttctccagaagaagtgggtggtaatgctctagtgcagttcggcacagaattggttttaccaatgccatttaaaggtgactggactcgccaagtacgccctgttctatttgctgaaggtggtcaagtatttgataccaagtgcgacgttcgatcttattctatgattatgaatggtcagcaaatttctgatgctaaaaaatactgtgaagataattatggatttgatttaggtaacttgcgttatagtgtgggtgtcggtgtaacctggattactatgattggtccattgtcacttagctatgcattcccgttaaatgataaaccgggtgatgaaactaaagaaattcagtttgaaattggtcgtactttctgataatag

SEQ ID NO: 11 - DNA, 1594 bp, synthetic construct
  misc_feature 1..1594: note = LOA-linker-A3B3-linker-MUC1 ORF
  source 1..1594: mol_type = other DNA
ggatccaaaataaggaggaaaaaaaatgaaagcgaccaaactggtgcttggcgccgtgattctgggtagcacccttctggccggttgtagcagtaatgcgaaaatcgatcagggcattaacccgtatgttggctttgaaatgggttacgactggttaggtcgtatgccgtacaaaggcagcgttgaaaacggtgcatacaaagctcagggcgttcaactgaccgctaaactgggttacccgatcactgacgacctggacatctacactcgtctgggtggcatggtatggcgtgcagacactaaatccaacgtttatggtaaaaaccacgacaccggcgtttctccggtcttcgctggcggtgttgagtacgcgatcactcctgaaatcgctacccgtctggaataccagtggaccaacaacatcggtgacgcacacaccatcggcactcgtccggacaacggcattccgggtgcggaagccgctgcaaaggaggctgccgcaaaagaagcagccgctaaggaggctgcggcaaaagcggctagcgtctctgcggagctgccgaagccttccatctccagcaacaactccaaaccggtggaggacaaggatgctgtggccttcacctgtgaacctgaggctcagaacacaacctacctttggtgggtaaatggtcagagcctgccggtcagtcctcgtctgcagctttccaatggcaaccgcaccctgactcttttcaatgtcacacgtaatgacgcacgcgcctatgtatgtggaatccagaactcagtgagtgcaaaccgcagtgaccctgtcaccctggatgtcctttatgggccggacaccccgatcatttcccctccggactcgtcttacctttcgggagcggatttaaacctgtcctgtcactcggcctctaacccttccccgcagtattcttggtttgtgaatgggacctttcagcaacacacacaagttatttttatcaccaaaatcacgcctaataataacgggacctatgcctgttttgtctctaacctggctactggccgcaataattccattgtcaagagcatcacagtcgcggaagccgctgcaaaggaggctgccgcaaaagaagcagccgctaaggaggctgcggcaaaagcgtctagaccggctcctggtagcacggcaccgcctgcgcatggtgtaacttctgctccggataatcgtcctgcgttaggaagtactgctcctccggctcacggggtgacgagtgcacctgacacgcgcccggcacctgggtctacagcgccgcctgcacatggagttacatccgcgccggataatcgtcctgctttgggatcgactgcaccgcctgcccatggggtaaccagtgcccctgatacccgtccggcacctggctcaacggcccctccggcacacggcgtcacgtcagcaccggacaaccgtcctgctttagggagcacagctccgcctgcgcatggagtcacttcagcgccggacacacgccctgccccgggctccaccgcccctccggcccacggtgtcacctcggcccctgacacccgtcctaggtaatgatagactagt

SEQ ID NO: 12 - protein, 518 aa, synthetic construct
  REGION 1..518: note = LOA-linker-A3B3-linker-MUC1
MKATKLVLGAVILGSTLLAGCSSNAKIDQGINPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDNGIPGAEAAAKEAAAKEAAAKEAAAKAASVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGADLNLSCHSASNPSPQYSWFVNGTFQQHTQVIFITKITPNNNGTYACFVSNLATGRNNSIVKSITVAEAAAKEAAAKEAAAKEAAAKASRPAPGSTAPPAHGVTSAPDNRPALGSTAPPAHGVTSAPDTRPAPGSTAPPAHGVTSAPDNRPALGSTAPPAHGVTSAPDTRPAPGSTAPPAHGVTSAPDNRPALGSTAPPAHGVTSAPDTRPAPGSTAPPAHGVTSAPDTRPR

SEQ ID NO: 13 - DNA, 1168 bp, synthetic construct
  misc_feature 1..1168: note = LOA-linker-A3B3-linker ORF
  source 1..1168: mol_type = other DNA
ggatccaaaataaggaggaaaaaaaatgaaagcgaccaaactggtgcttggcgccgtgattctgggtagcacccttctggccggttgtagcagtaatgcgaaaatcgatcagggcattaacccgtatgttggctttgaaatgggttacgactggttaggtcgtatgccgtacaaaggcagcgttgaaaacggtgcatacaaagctcagggcgttcaactgaccgctaaactgggttacccgatcactgacgacctggacatctacactcgtctgggtggcatggtatggcgtgcagacactaaatccaacgtttatggtaaaaaccacgacaccggcgtttctccggtcttcgctggcggtgttgagtacgcgatcactcctgaaatcgctacccgtctggaataccagtggaccaacaacatcggtgacgcacacaccatcggcactcgtccggacaacggcattccgggtgcggaagccgctgcaaaggaggctgccgcaaaagaagcagccgctaaggaggctgcggcaaaagcggctagcgtctctgcggagctgccgaagccttccatctccagcaacaactccaaaccggtggaggacaaggatgctgtggccttcacctgtgaacctgaggctcagaacacaacctacctttggtgggtaaatggtcagagcctgccggtcagtcctcgtctgcagctttccaatggcaaccgcaccctgactcttttcaatgtcacacgtaatgacgcacgcgcctatgtatgtggaatccagaactcagtgagtgcaaaccgcagtgaccctgtcaccctggatgtcctttatgggccggacaccccgatcatttcccctccggactcgtcttacctttcgggagcggatttaaacctgtcctgtcactcggcctctaacccttccccgcagtattcttggtttgtgaatgggacctttcagcaacacacacaagttatttttatcaccaaaatcacgcctaataataacgggacctatgcctgttttgtctctaacctggctactggccgcaataattccattgtcaagagcatcacagtcgcggaagccgctgcaaaggaggctgccgcaaaagaagcagccgctaaggaggctgcggcaaaagcgtctaggtaatgatagactagt

SEQ ID NO: 14 - protein, 376 aa, synthetic construct
  REGION 1..376: note = LOA-linker-A3B3-linker
MKATKLVLGAVILGSTLLAGCSSNAKIDQGINPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDNGIPGAEAAAKEAAAKEAAAKEAAAKAASVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGADLNLSCHSASNPSPQYSWFVNGTFQQHTQVIFITKITPNNNGTYACFVSNLATGRNNSIVKSITVAEAAAKEAAAKEAAAKEAAAKASR

SEQ ID NO: 15 - DNA, 921 bp, synthetic construct
  misc_feature 1..921: note = ClyA
  source 1..921: mol_type = other DNA
atgactagtatttttgcagaacaaactgtagaggtagttaaaagcgcgatcgaaaccgcagatggggcattagatctttataacaaatacctcgaccaggtcatcccctggaagacctttgatgaaaccataaaagagttaagccgttttaaacaggagtactcgcaggaagcttctgttttagttggtgatattaaagttttgcttatggacagccaggacaagtattttgaagcgacacaaactgtttatgaatggtgtggtgtcgtgacgcaattactctcagcgtatattttactatttgatgaatataatgagaaaaaagcatcagcccagaaagacattctcattaggatattagatgatggtgtcaagaaactgaatgaagcgcaaaaatctctcctgacaagttcacaaagtttcaacaacgcttccggaaaactgctggcattagatagccagttaactaatgatttttcggaaaaaagtagttatttccagtcacaggtggatagaattcgtaaggaagcttatgccggtgctgcagccggcatagtcgccggtccgtttggattaattatttcctattctattgctgcgggcgtgattgaagggaaattgattccagaattgaataacaggctaaaaacagtgcaaaatttctttactagcttatcagctacagtgaaacaagcgaataaagatatcgatgcggcaaaattgaaattagccactgaaatagcagcaattggggagataaaaacggaaaccgaaacaaccagattctacgttgattatgatgatttaatgctttctttattaaaaggagctgcaaagaaaatgattaacacctgtaatgaataccaacaacgtcatggtaagaagacgcttttcgaggttcctgacgtcgctagctgataa

SEQ ID NO: 16 - protein, 305 aa, Salmonella enterica
MTSIFAEQTVEVVKSAIETADGALDLYNKYLDQVIPWKTFDETIKELSRFKQEYSQEASVLVGDIKVLLMDSQDKYFEATQTVYEWCGVVTQLLSAYILLFDEYNEKKASAQKDILIRILDDGVKKLNEAQKSLLTSSQSFNNASGKLLALDSQLTNDFSEKSSYFQSQVDRIRKEAYAGAAAGIVAGPFGLIISYSIAAGVIEGKLIPELNNRLKTVQNFFTSLSATVKQANKDIDAAKLKLATEIAAIGEIKTETETTRFYVDYDDLMLSLLKGAAKKMINTCNEYQQRHGKKTLFEVPDVAS

SEQ ID NO: 17 - DNA, 985 bp, synthetic construct
  misc_feature 1..985: note = LOA-linker- MUC1 ORF
  source 1..985: mol_type = other DNA
ggatccaaaataaggaggaaaaaaaatgaaagcgaccaaactggtgcttggcgccgtgattctgggtagcacccttctggccggttgtagcagtaatgcgaaaatcgatcagggcattaacccgtatgttggctttgaaatgggttacgactggttaggtcgtatgccgtacaaaggcagcgttgaaaacggtgcatacaaagctcagggcgttcaactgaccgctaaactgggttacccgatcactgacgacctggacatctacactcgtctgggtggcatggtatggcgtgcagacactaaatccaacgtttatggtaaaaaccacgacaccggcgtttctccggtcttcgctggcggtgttgagtacgcgatcactcctgaaatcgctacccgtctggaataccagtggaccaacaacatcggtgacgcacacaccatcggcactcgtccggacaacggcattccgggtgcggaagccgctgcaaaggaggctgccgcaaaagaagcagccgctaaggaggctgcggcaaaagcggctagaccggctcctggtagcacggcaccgcctgcgcatggtgtaacttctgctccggataatcgtcctgcgttaggaagtactgctcctccggctcacggggtgacgagtgcacctgacacgcgcccggcacctgggtctacagcgccgcctgcacatggagttacatccgcgccggataatcgtcctgctttgggatcgactgcaccgcctgcccatggggtaaccagtgcccctgatacccgtccggcacctggctcaacggcccctccggcacacggcgtcacgtcagcaccggacaaccgtcctgctttagggagcacagctccgcctgcgcatggagtcacttcagcgccggacacacgccctgccccgggctccaccgcccctccggcccacggtgtcacctcggcccctgacacccgtcctaggtaatgatagactagt

SEQ ID NO: 18 - protein, 315 aa, synthetic construct
  REGION 1..315: note = LOA-linker- MUC1
MKATKLVLGAVILGSTLLAGCSSNAKIDQGINPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDNGIPGAEAAAKEAAAKEAAAKEAAAKAARPAPGSTAPPAHGVTSAPDNRPALGSTAPPAHGVTSAPDTRPAPGSTAPPAHGVTSAPDNRPALGSTAPPAHGVTSAPDTRPAPGSTAPPAHGVTSAPDNRPALGSTAPPAHGVTSAPDTRPAPGSTAPPAHGVTSAPDTRPR

SEQ ID NO: 19 - DNA, 2062 bp, synthetic construct
  misc_feature 1..2062: note = I198N-linker-A3B3-linker-MUC1
  source 1..2062: mol_type = other DNA
ggatccaaaataaggaggaaaaaaaatgatttttgcagaacaaactgtagaggtagttaaaagcgcgatcgaaaccgcagatggggcattagatctttataacaaatacctggaccaggtcatcccgtggaagacctttgatgaaaccattaaagagttaagccgttttaaacaggagtactcgcaggaagcttctgttttagttggtgatattaaagttttgcttatggacagccaggacaagtattttgaagcgacacaaactgtttatgaatggtgtggtgtcgtgacgcaattactgtcagcgtatattttactttttgatgaatataatgagaaaaaagcatcagcccagaaagacattctgatccgtattttagatgatggtgtcaagaaactgaatgaagcgcaaaaatctcttctgacaagttcacaaagtttcaacaacgcttccggaaaactgcttgcattagatagccagctgactaatgatttttcggaaaaaagtagttatttccagtcacaggtggatcgcattcgtaaggaagcttatgccggtgctgcagccggcatcgtcgccggtccgtttggattaattatctcctattctaatgctgcgggcgtgattgaagggaaattgattccggaactgaataaccgtcttaaaacagtgcaaaatttctttactagcttatcagctacagtgaaacaagcgaataaagatatcgatgcggcaaaattgaaattagccactgaaatcgcagcaatcggggagatcaaaacggaaaccgaaacaacccgcttctacgttgattatgatgatttaatgctttctttgttaaaaggagctgcaaagaaaatgattaacacctgtaatgaataccaacaacgtcatggtaagaagacgcttttcgaggttcctgacgtcggcattccgggtgcggaagccgctgcaaaggaggctgccgcaaaagaagcagccgctaaggaggctgcggcaaaagcggctagcgtctctgcggagctgccgaagccttccatctccagcaacaactccaaaccggtggaggacaaggatgctgtggccttcacctgtgaacctgaggctcagaacacaacctacctttggtgggtaaatggtcagagcctgccggtcagtcctcgtctgcagctttccaatggcaaccgcaccctgactcttttcaatgtcacacgtaatgacgcacgcgcctatgtatgtggaatccagaactcagtgagtgcaaaccgcagtgaccctgtcaccctggatgtcctttatgggccggacaccccgatcatttcccctccggactcgtcttacctttcgggagcggatttaaacctgtcctgtcactcggcctctaacccttccccgcagtattcttggtttgtgaatgggacctttcagcaacacacacaagttatttttatcaccaaaatcacgcctaataataacgggacctatgcctgttttgtctctaacctggctactggccgcaataattccattgtcaagagcatcacagtcgcggaagccgctgcaaaggaggctgccgcaaaagaagcagccgctaaggaggctgcggcaaaagcgtctagaccggctcctggtagcacggcaccgcctgcgcatggtgtaacttctgctccggataatcgtcctgcgttaggaagtactgctcctccggctcacggggtgacgagtgcacctgacacgcgcccggcacctgggtctacagcgccgcctgcacatggagttacatccgcgccggataatcgtcctgctttgggatcgactgcaccgcctgcccatggggtaaccagtgcccctgatacccgtccggcacctggctcaacggcccctccggcacacggcgtcacgtcagcaccggacaaccgtcctgctttagggagcacagctccgcctgcgcatggagtcacttcagcgccggacacacgccctgccccgggctccaccgcccctccggcccacggtgtcacctcggcccctgacacccgtcctaggtaatgatagactagt

SEQ ID NO: 20 - protein, 674 aa, synthetic construct
  REGION 1..674: note = I198N-linker-A3B3-linker-MUC1
MIFAEQTVEVVKSAIETADGALDLYNKYLDQVIPWKTFDETIKELSRFKQEYSQEASVLVGDIKVLLMDSQDKYFEATQTVYEWCGVVTQLLSAYILLFDEYNEKKASAQKDILIRILDDGVKKLNEAQKSLLTSSQSFNNASGKLLALDSQLTNDFSEKSSYFQSQVDRIRKEAYAGAAAGIVAGPFGLIISYSNAAGVIEGKLIPELNNRLKTVQNFFTSLSATVKQANKDIDAAKLKLATEIAAIGEIKTETETTRFYVDYDDLMLSLLKGAAKKMINTCNEYQQRHGKKTLFEVPDVGIPGAEAAAKEAAAKEAAAKEAAAKAASVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGADLNLSCHSASNPSPQYSWFVNGTFQQHTQVIFITKITPNNNGTYACFVSNLATGRNNSIVKSITVAEAAAKEAAAKEAAAKEAAAKASRPAPGSTAPPAHGVTSAPDNRPALGSTAPPAHGVTSAPDTRPAPGSTAPPAHGVTSAPDNRPALGSTAPPAHGVTSAPDTRPAPGSTAPPAHGVTSAPDNRPALGSTAPPAHGVTSAPDTRPAPGSTAPPAHGVTSAPDTRPR

SEQ ID NO: 21 - protein, 145 aa, synthetic construct
  REGION 1..145: note = lpp-ompA
MKATKLVLGAVILGSTLLAGCSSNAKIDQGINPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDN

SEQ ID NO: 22 - protein, 21 aa, synthetic construct
  REGION 1..21: note = linker sequence
AEAAAKEAAAKEAAAKEAAAK

SEQ ID NO: 23 - protein, 179 aa, synthetic construct
  REGION 1..179: note = CEA A3B3
VSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGADLNLSCHSASNPSPQYSWFVNGTFQQHTQVIFITKITPNNNGTYACFVSNLATGRNNSIVKSITV

SEQ ID NO: 24 - protein, 142 aa, synthetic construct
  REGION 1..142: note = MUC-1
PAPGSTAPPAHGVTSAPDNRPALGSTAPPAHGVTSAPDTRPAPGSTAPPAHGVTSAPDNRPALGSTAPPAHGVTSAPDTRPAPGSTAPPAHGVTSAPDNRPALGSTAPPAHGVTSAPDTRPAPGSTAPPAHGVTSAPDTRPR

SEQ ID NO: 25 - protein, 301 aa, synthetic construct
  REGION 1..301: note = ClyAI198N
MIFAEQTVEVVKSAIETADGALDLYNKYLDQVIPWKTFDETIKELSRFKQEYSQEASVLVGDIKVLLMDSQDKYFEATQTVYEWCGVVTQLLSAYILLFDEYNEKKASAQKDILIRILDDGVKKLNEAQKSLLTSSQSFNNASGKLLALDSQLTNDFSEKSSYFQSQVDRIRKEAYAGAAAGIVAGPFGLIISYSNAAGVIEGKLIPELNNRLKTVQNFFTSLSATVKQANKDIDAAKLKLATEIAAIGEIKTETETTRFYVDYDDLMLSLLKGAAKKMINTCNEYQQRHGKKTLFEVPDV

SEQ ID NO: 26 - DNA, 745 bp, synthetic construct
  misc_feature 1..745: note = Francisella novicida (lpxEFn ) synthetic gene sequence
  source 1..745: mol_type = other DNA
aaaataaggaggaaaaaaaatgctgaaacagaccctgcaaacgaactttcagggttttaaagatatttttaagaaaccgaaactacacaatcataaattgcctcgctatctacagttgaaatatacgtttataccattattaattttggtaatttttgcatactataacttagataccccagttgagaactatatcaagcattctatgccaaatattgttggtgtaatttttggtaaaataactgatgttggtaaggccgagtatattttgataatttgcggtgtgatagtgttagcgcgtttatttacagatagccaaaaattatctgctaatactcgcgctatgtttgacaaggtgtcggcatatgcgggttttatcttagcaactgtagctattagtggtattttgggacaaatactcaagatgataataggtcgcgcgcgtcctaagtttttcttggaatatggttcgcattatttccaacattttcatgcacctggatatgattttgcaagtatgccgtcagggcactcaatcacagttggagcaatgtttatagcatttttttatattttccctaagctacgctatttttggtatttgctgatagtggtatttgctgggagtcgtattatggttggttcacattatcctagtgatgtaatttttggcgttgcttttggttgttactgtacagcatatatctactattggatgcgcaatcgtgagattatttagtgataa

SEQ ID NO: 27 - protein, 239 aa, Francisella novicida
MLKQTLQTNFQGFKDIFKKPKLHNHKLPRYLQLKYTFIPLLILVIFAYYNLDTPVENYIKHSMPNIVGVIFGKITDVGKAEYILIICGVIVLARLFTDSQKLSANTRAMFDKVSAYAGFILATVAISGILGQILKMIIGRARPKFFLEYGSHYFQHFHAPGYDFASMPSGHSITVGAMFIAFFYIFPKLRYFWYLLIVVFAGSRIMVGSHYPSDVIFGVAFGCYCTAYIYYWMRNREII

SEQ ID NO: 28 - DNA, 435 bp, synthetic construct
  misc_feature 1..435: note = lpp-ompA sequence
  source 1..435: mol_type = other DNA
atgaaagcgaccaaactggtgcttggcgccgtgattctgggtagcacccttctggccggttgtagcagtaatgcgaaaatcgatcagggcattaacccgtatgttggctttgaaatgggttacgactggttaggtcgtatgccgtacaaaggcagcgttgaaaacggtgcatacaaagctcagggcgttcaactgaccgctaaactgggttacccgatcactgacgacctggacatctacactcgtctgggtggcatggtatggcgtgcagacactaaatccaacgtttatggtaaaaaccacgacaccggcgtttctccggtcttcgctggcggtgttgagtacgcgatcactcctgaaatcgctacccgtctggaataccagtggaccaacaacatcggtgacgcacacaccatcggcactcgtccggacaac

SEQ ID NO: 29 - DNA, 1521 bp, Salmonella enterica
  source 1..1521: mol_type = other DNA
atggcacaagtcattaatacaaacagcctgtcgctgttgacccagaataacctgaacaaatcccagtccgcactgggcactgctatcgagcgtttgtcttccggtctgcgtatcaacagcgcgaaagacgatgcggcaggacaggcgattgctaaccgttttaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgcgaatggtactaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggtgccaacgacggtgaaactatcgatattgatttaaaagaaatcagctctaaaacactgggacttgataagcttaatgtccaagatgcctacaccccgaaagaaactgctgtaaccgttgataaaactacctataaaaatggtacagatcctattacagcccagagcaatactgatatccaaactgcaattggcggtggtgcaacgggggttactggggctgatatcaaatttaaagatggtcaatactatttagatgttaaaggcggtgcttctgctggtgtttataaagccacttatgatgaaactacaaagaaagttaatattgatacgactgataaaactccgttggcaactgcggaagctacagctattcggggaacggccactataacccacaaccaaattgctgaagtaacaaaagagggtgttgatacgaccacagttgcggctcaacttgctgcagcaggggttactggcgccgataaggacaatactagccttgtaaaactatcgtttgaggataaaaacggtaaggttattgatggtggctatgcagtgaaaatgggcgacgatttctatgccgctacatatgatgagaaaacaggtgcaattactgctaaaaccactacttatacagatggtactggcgttgctcaaactggagctgtgaaatttggtggcgcaaatggtaaatctgaagttgttactgctaccgatggtaagacttacttagcaagcgaccttgacaaacataacttcagaacaggcggtgagcttaaagaggttaatacagataagactgaaaacccactgcagaaaattgatgctgccttggcacaggttgatacacttcgttctgacctgggtgcggttcagaaccgtttcaactccgctatcaccaacctgggcaataccgtaaataacctgtcttctgcccgtagccgtatcgaagattccgactacgcaaccgaagtctccaacatgtctcgcgcgcagattctgcagcaggccggtacctccgttctggcgcaggcgaaccaggttccgcaaaacgtcctctctttactgcgttaa

SEQ ID NO: 30 - DNA, 537 bp, synthetic construct
  misc_feature 1..537: note = A3B3
  source 1..537: mol_type = other DNA
gtctctgcggagctgccgaagccttccatctccagcaacaactccaaaccggtggaggacaaggatgctgtggccttcacctgtgaacctgaggctcagaacacaacctacctttggtgggtaaatggtcagagcctgccggtcagtcctcgtctgcagctttccaatggcaaccgcaccctgactcttttcaatgtcacacgtaatgacgcacgcgcctatgtatgtggaatccagaactcagtgagtgcaaaccgcagtgaccctgtcaccctggatgtcctttatgggccggacaccccgatcatttcccctccggactcgtcttacctttcgggagcggatttaaacctgtcctgtcactcggcctctaacccttccccgcagtattcttggtttgtgaatgggacctttcagcaacacacacaagttatttttatcaccaaaatcacgcctaataataacgggacctatgcctgttttgtctctaacctggctactggccgcaataattccattgtcaagagcatcacagtc

SEQ ID NO: 31 - DNA, 426 bp, synthetic construct
  misc_feature 1..426: note = MUC-1
  source 1..426: mol_type = other DNA
ccggctcctggtagcacggcaccgcctgcgcatggtgtaacttctgctccggataatcgtcctgcgttaggaagtactgctcctccggctcacggggtgacgagtgcacctgacacgcgcccggcacctgggtctacagcgccgcctgcacatggagttacatccgcgccggataatcgtcctgctttgggatcgactgcaccgcctgcccatggggtaaccagtgcccctgatacccgtccggcacctggctcaacggcccctccggcacacggcgtcacgtcagcaccggacaaccgtcctgctttagggagcacagctccgcctgcgcatggagtcacttcagcgccggacacacgccctgccccgggctccaccgcccctccggcccacggtgtcacctcggcccctgacacccgtcctagg